(19) United States
(12) Patent Application Publication
(10) Pub. No.: US 2003/0220376 A1
(43) Pub. Date: Nov. 27, 2003

(54) Methods for treating carbonic anhydrase mediated disorders

(75) Inventors: Jaime L. Masferrer, Ballwin, MO (US); Janet M. O'Neal, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/367,384

(22) Filed: Feb. 14, 2003

Related U.S. Application Data
(63) Continuation-in-part of application No. 10/213,793, filed on Aug. 7, 2002.
(60) Provisional application No. 60/311,561, filed on Aug. 10, 2001.

(51) Int. Cl. A61K 31/41; A61K 31/429; A61K 31/4245; A61K 31/426; A61K 31/421; A61K 31/4196; A61K 31/4192

(52) U.S. Cl. 514/359; 514/362; 514/364; 514/383; 514/424; 514/407; 514/369; 514/376

(57) ABSTRACT

The current invention provides methods to treat or prevent carbonic anhydrase mediated diseases or disorders. The method generally comprises administering a tricyclic compound having a sulfonamide group to a subject wherein the compound inhibits carbonic anhydrase.

### CROSS REFERENCE TO RELATED APPLICATIONS

This application is a continuation in part of application Ser. No. 10/213,793 filed on Aug. 7, 2002, which claims priority from provisional application Serial No. 60/311,561 filed on Aug. 10, 2001, both of which are hereby incorporated by reference in their entirety.

### FIELD OF THE INVENTION

The present invention generally provides methods to treat or prevent a carbonic anhydrase mediated disorder in a subject. More specifically, the method comprises administering to the subject a compound having a sulfonamide group that inhibits carbonic anhydrase.

### BACKGROUND OF THE INVENTION

Carbonic anhydrase (CA), also called carbonate dehydratase, catalyzes the hydration of carbon dioxide in the reaction CO₂+H₂O→HCO⁻₃+H⁺. CA is highly ubiquitous in nature (e.g. being present in animals, plants and certain bacteria) and may exist as a number of different isoforms within the same species. For example, eight enzymatic and evolutionarily related forms of CA are currently known to exist in humans: three cytosolic isozymes (CAI, CAII, and CAIII), two membrane-bound forms (CAIV and CAVII), a mitochondrial form (CAV), a secreted salivary form (CAVI) and an isozyme that has not presently been characterized.

CA is crucial for a number of physiological functions. In one such function, CA is necessary to maintain pH homeostasis. One means by which the body maintains pH homeostasis, despite the constant influx of acids from both dietary and metabolic sources, is through the use of a number of intracellular and extracellular buffers, such as HCO₃⁻. By catalyzing the breakdown of CO₂ and H₂O, CA helps maintain the body's buffering capacity by regenerating HCO₃⁻. Moreover, in the kidneys, the reabsorption of HCO₃⁻ in the renal proximal tubule is catalyzed by CA, which combines CO₂ with the OH⁻ ion that results from the splitting of water. CA is also one of the key enzymes responsible for electrolyte secretion in a variety of tissues. By way of example, CA in the extracellular boundary layer of sarcolemma facilitates CO₂ transport via the catalyzed hydration of CO₂, thus maintaining the PCO₂ gradient across the sarcolemma, and H⁺ released in that reaction protonates excreted NH₃⁻ which helps maintain the PNH₃ gradient (Henry, R. P. et al. (1997) Am. J. Physiol. 262(6/2):R1754-R1761). By way of further example, in the placenta, CA may provide ions for exchange with Na⁺, K⁺, and Cl⁻ in transepithelial movement of ions and fluid, as well as facilitating CO₂ diffusion. CA can also be active in intermediary metabolism, such as glucorieogenesis, lipogenesis, ureagenesis and fatty acid synthesis (Ridderstrale, Y. (1997) Microsc. Res. Tech. 38(1-2):115-124).

A number of disease states are marked by variations in CA activity. The concentration of CAII in the cerebrospinal fluid (CSF) appears to mark disease activity in patients with brain damage. Moreover, high CA concentrations have been observed in patients with brain infarction. Patients with transient ischemic attack, multiple sclerosis, or epilepsy usually have CAII concentrations in the normal range, but higher CAII levels have been observed in the CSF of those with central nervous system infection, dementia, or trigeminal neuralgia (Parkkila, A. K. et al. (1997) Eur. J. Clin. Invest. 27(5):392-397). Colonic adenomas and adenocarcinomas have been observed to fail to stain for CA, whereas non-neoplastic controls showed CAI and CAI in the cytoplasm of the columnar cells lining the upper half of colonic crypts. The neoplasms show staining patterns similar to less mature cells lining the base of normal crypts (Gramlich T. L. et al. (1990) Arch. Pathol. Lab. Med. 114(4):415-419). Deficiency of CAII has also been identified as the primary defect in osteopetrosis, a rare metabolic bone disease characterized by increase in skeletal mass due to a defect in development or function of the osteoclasts (Felix, R. et al. (1996) Eur. J. Endocrinol. 134(2):143-156).

Therapeutic interventions in a number of diseases involve altering CA activity. Ophthalmic disorders are commonly treated with carbonic anhydrase inhibitors such as acetazolamide. Carbonic anhydrase inhibitors are also used to treat chronic renal failure (Suki, W. N. (1997) Kidney Int. Suppl. 59:S33-S35), Parkinson's Disease and tardive dyskinesia (Cowen, M. A. et al. (1997) J. Clin. Pharmacol. 17(3):190-193), and epileptic seizures uncontrolled by other marketed agents (Reiss, W. G. (1996) Ann. Pharmacother. 30(5):514-519). Moreover, inhibition of CA has also been shown to be an effective treatment for several types of neoplasia. For example, sulfonamide carbonic anhydrase inhibitors have been shown to inhibit cell growth in leukemia, non-small cell lung cancer, ovarian cancer, melanoma, colon, CNS, renal, prostate and breast cancer cell lines. (C. Supuran,et al, Eur.J.Med.Chem, 35: 867-874 (2000)).

### SUMMARY OF THE INVENTION

Among the several aspects of the invention is the provision of a method to treat carbonic anhydrase mediated disorders in a subject. Briefly, the method comprises administering to a subject a carbonic anhydrase inhibitor having a sulfonamide group. The method may be employed to treat a number of different carbonic anhydrase mediated disorders including elevated intraocular pressure, edema, altitude sickness, periodic paralysis, cystine calculi, and uric acid calculi.

In one embodiment, the method comprises administering to a subject a carbonic anhydrase inhibitor or pharmaceutically acceptable salt or prodrug thereof corresponding to formula (I)

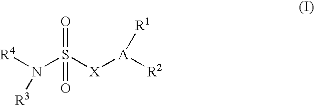

wherein:

X comprises a 5- or 6-membered heterocyclic or carbocyclic ring, the ring atoms being X₁, X₂, X₃, X₄, and X₅ for 5-membered rings and X₁, X₂, X₃, X₄, X₅ and X₆ for 6-membered rings, wherein X₂ is alpha to each of X₁ and X₃, X₃ is alpha to each of X₂ and X₄, X₄ is alpha to each of X₃ and X₅, X₅ is alpha to X₄ and alpha to X₁ if X is a 5-membered ring or to X₆ if X is a 6-membered ring, and X₆, when present, is alpha to each of X₁ and X₅, wherein X₁, X₂, X₃, X₄, X₅ and X₆ are carbon, nitrogen, oxygen or sulfur;

A is selected from the group consisting of a heterocyclic ring or a carbocyclic ring;

R¹ is selected from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R¹ is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo or wherein R¹ together with ring A forms one or more heterocyclyl or carbocyclic rings where the heterocyclyl or carbocyclic ring is optionally substituted with a substituent selected from the group consisting of oxo, acyl, hydrocarbyl, substituted hydrocarbyl, cyano, carboxyl, hydroxyl, amino, nitro, aminocarbonyl, aminosulfonyl and halo;

R² is selected from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R² is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo;

R³ is selected from the group consisting of hydrogen, hydrocarbyl and substituted hydrocarbyl; and

R⁴ is selected from the group consisting of hydrogen, hydrocarbyl and substituted hydrocarbyl.

In yet another embodiment, the method comprises administering to a subject a carbonic anhydrase inhibitor or pharmaceutically acceptable salt or prodrug thereof corresponding to formula (III)

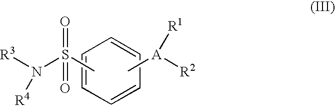

wherein:

A is selected from the group consisting of a heterocyclic ring or a carbocyclic ring;

R¹ is selected from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R¹ is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo or wherein R¹ together with ring A forms one or more heterocyclyl or carbocyclic rings where the heterocyclyl or carbocyclic ring is optionally substituted with a substituent selected from the group consisting of oxo, acyl, hydrocarbyl, substituted hydrocarbyl, cyano, carboxyl, hydroxyl, amino, nitro, aminocarbonyl, aminosulfonyl and halo;

R² is selected from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R² is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo;

R³ is selected from the group consisting of hydrogen, hydrocarbyl and substituted hydrocarbyl; and

R⁴ is selected from the group consisting of hydrogen, hydrocarbyl and substituted hydrocarbyl.

In still another embodiment, the method comprises administering to a subject a carbonic anhydrase inhibitor or pharmaceutically acceptable salt or prodrug thereof corresponding to formula (V)

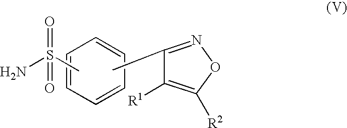

wherein:

R¹ is selected from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R¹ is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo or wherein R¹ together with the pyrazolyl ring forms one or more heterocyclyl or carbocyclic rings where the heterocyclyl or carbocyclic ring is optionally substituted with a substituent selected from the group consisting of oxo, acyl, hydrocarbyl, substituted hydrocarbyl, cyano, carboxyl, hydroxyl, amino, nitro, aminocarbonyl, aminosulfonyl and halo; and

R² is selected from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R² is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo.

In a further embodiment, the method comprises administering to a subject a carbonic anhydrase inhibitor or pharmaceutically acceptable salt or prodrug thereof corresponding to formula (VI)

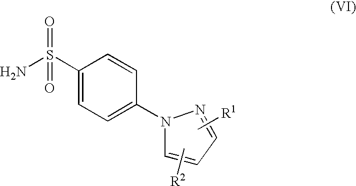

wherein:

R¹ is selected from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R¹ is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo or wherein R¹ together with the pyrazolyl ring forms one or more heterocyclyl or carbocyclic rings where the heterocyclyl or carbocyclic ring is optionally substituted with a substituent selected from the group consisting of oxo, acyl, hydrocarbyl, substituted hydrocarbyl, cyano, carboxyl, hydroxyl, amino, nitro, aminocarbonyl, aminosulfonyl and halo; and

R² is selected from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R² is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo.

Other features and aspects of the invention are described more thoroughly below.

Abbreviations and Definitions

The term “acyl” is a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, trifluoroacetyl.

The term “alkenyl” is a linear or branched radical having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, butenyl and 4-methylbutenyl.

The terms “alkenyl” and “lower alkenyl,” are radicals having “cis” and “trans” orientations, or alternatively, “E” and “Z” orientations.

The term “alkoxycarbonyl” means a radical containing an alkoxy radical, as defined above, attached via an oxygen atom to a carbonyl radical. More preferred are “lower alkoxycarbonyl” radicals with alkyl portions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

The term “alkylamino” is an amino group that has been substituted with one or two alkyl radicals. Preferred are “lower N-alkyl” radicals having alkyl portions having 1 to 6 carbon atoms. Suitable lower alkylamino may be mono or dialkylamino such as N-methylamino, N-ethylamino, N,N-dimethylamino, N,N-diethylamino or the like.

The term “alkylaminoalkyl” is a radical having one or more alkyl radicals attached to an aminoalkyl radical.

The term “alkylaminocarbonyl” is an aminocarbonyl group that has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “alkylsulfinyl” is a radical containing a linear or branched alkyl radicals, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “alkylthio” is a radical containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkylthioalkyl” is a radical containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkynyl” is a linear or branched radical having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The term “aminoalkyl” is an alkyl radical substituted with one or more amino radical. More preferred are “lower aminoalkyl” radicals. Examples of such radicals include aminomethyl, aminoethyl, and the like.

The term “aminocarbonyl” is an amide group of the formula —C(═O)NH₂.

The term “aralkoxy” is an aralkyl radical attached through an oxygen atom to other radicals.

The term “aralkoxyalkyl” is an aralkoxy radical attached through an oxygen atom to an alkyl radical.

The term “aralkyl” is an aryl-substituted alkyl radical such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy. The terms benzyl and phenylmethyl are interchangeable.

The term “aralkylamino” is an aralkyl radical attached through an amino nitrogen atom to other radicals.

The term “aralkylthio” is an aralkyl radical attached to a sulfur atom.

The term “aralkylthioalkyl” is an aralkylthio radicals attached through a sulfur atom to an alkyl radical.

The term “aroyl” is an aryl radical with a carbonyl radical as defined above. Examples of aroyl include benzoyl, naphthoyl, and the like and the aryl in said aroyl may be additionally substituted.

The term “aryl” is amino groups, which have been substituted with one or two aryl radicals, such as N-phenylamino. The “aryl” radicals may be further substituted on the aryl ring portion of the radical.

The term “aryloxyalkyl” is a radical having an aryl radical attached to an alkyl radical through a divalent oxygen atom.

The term “arylthioalk” is a radical having an aryl radical attached to an alkyl radical through a divalent sulfur atom.

The term “carbonic anhydrase” as used herein refers to any isoform of the metalloprotein enzyme that catalyzes the interconversion of CO₂and H₂CO₃ (CO₂+O₂→HCO₂⁻+H⁺).

The term “cycloalkyl” is a saturated carbocyclic radical having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The term “cycloalkenyl” is a partially unsaturated carbocyclic radical having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl.

The terms “cyclooxygenase-1” and “COX-1” used interchangeably herein refer to the constitutive isoform of the enzyme cyclooxygenase.

The terms “cyclooxygenase-2” and “COX-2 as used interchangeably herein refer to the inducible isoform of the enzyme cyclooxygenase. As used herein, the terms “cyclooxygenase-2 selective inhibitor” and “COX-2 selective inhibitor” are used interchangeably herein refer to a therapeutic compound that inhibits cyclooxygenase-2 more than it inhibits cyclooxygenase-1. The term “cyclooxygenase-2 inhibitor” or “COX-2 inhibitor” refers to any compound that inhibits the COX-2 enzyme, without regard to the extent to which it inhibits COX-1. Suitable cyclooxygenase-2 selective inhibitors useful in the present invention are those compounds that have a cyclooxygenase-2 IC₅₀ of less than about 0.2 μM, and also have a selectivity ratio of cyclooxygenase-2 inhibition over cyclooxygenase-1 inhibition of at least 50, and more preferably of at least 100. Even more preferably, the cyclooxygenase-2 selective inhibitor compounds have a cyclooxygenase-₁ IC₅₀ of greater than about 1 μM, and more preferably of greater than 10 μM.

The term “halo” means halogens such as fluorine, chlorine, bromine or iodine.

The term “haloalkyl” is a radical wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” are radicals having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

Where used, either alone or within other terms such as “haloalkyl”, “alkylsulfonyl”, “alkoxyalkyl” and “hydroxyalkyl”, the term “alkyl” is a linear, cyclic or branched radical having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “heteroaryl” is an unsaturated heterocyclyl radical. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also is a radical where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino.

The term “heterocyclyl” is a saturated, partially unsaturated and unsaturated heteroatom-containing ring-shaped radical, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The term “heterocyclylalkyl” is a saturated and partially unsaturated heterocyclyl-substituted alkyl radical, such as pyrrolidinylmethyl, and heteroaryl-substituted alkyl radical, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The term “hydrido” is a single hydrogen atom (H). This hydrido radical may be attached, for example, to an oxygen atom to form a hydroxyl radical or two hydrido radicals may be attached to a carbon atom to form a methylene (—CH₂—) radical.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “hydroxyalkyl” is a linear or branched alkyl radical having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The term “inhibition” as used herein means decrease the severity of an evident carbonic anhydrase mediated disorder as compared to that which would occur in the absence of the administration of a compound having any of formulas (I)-(VII).

The term “inhibitor” when used herein unless otherwise indicated refers to an enzyme inhibitor such as an inhibitor of carbonic anhydrase or cyclooxygenase. Enzyme inhibitors are agents and/or compounds that stop, prevent, or reduce the rate of an enzymatic reaction via any mechanism including, but not limited to, competitive inhibition, noncompetitive inhibition, and uncompetitive inhibition.

The terms “N-arylaminoalkyl” and “N-aryl-N-alkyl-aminoalkyl” are amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “prevention” includes either preventing the onset of a clinically evident carbonic anhydrase mediated disorder altogether or preventing the onset of a preclinically evident stage of a carbonic anhydrase mediated disorder in a subject. This definition includes prophylactic treatment.

The term “subject” for purposes of treatment or prevention includes any human or animal subject who is susceptible to a carbonic anhydrase mediated disorder. The subject can be a domestic livestock species, a farm animal, a laboratory animal species, a zoo animal or a companion animal. In one embodiment, the subject is a mammal. In an alternative of this embodiment, the mammal is a human being.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorus, boron, sulfur, or a halogen atom. Exemplary substituted hydrocarbyl moieties include, heterocyclo, alkoxyalkyl, alkenyloxyalkyl, alkynyloxyalkyl, aryloxyalkyl, hydroxyalkyl, protected hydroxyalkyl, keto, acyl, nitroalkyl, aminoalkyl, cyano, alkylthioalkyl, arylthioalkyl, ketals, acetals, amides, acids, esters and the like.

The term “sulfonyl”, whether used alone or linked to other terms such as alkylsulfonyl, is divalent radicals—SO₂—. “Alkylsulfonyl” is a alkyl radical attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” are NH₂O₂S—.

The phrase “therapeutically-effective” is intended to qualify the amount of compound having any of formulas (I)-(VII) that will achieve the goal of improvement in disorder severity and the frequency of incidence over no treatment, while avoiding adverse side effects typically associated with alternative therapies.

Description of the Preferred Embodiments

The present invention provides a method to treat or prevent carbonic anhydrase mediated disorders. Generally speaking, the method comprises administering a carbonic anhydrase inhibitor to a subject for the treatment or prevention of a number of carbonic anhydrase mediated disorders including elevated intraocular pressure, edema, altitude sickness, periodic paralysis, cystine calculi, and uric acid calculi. In addition to inhibiting carbonic anhydrase, several of the compounds employed in the present invention also selectively inhibit cyclooxygenase-2. Accordingly, these compounds may also be advantageously employed for indications benefited by the inhibition of both carbonic anhydrase and cyclooxygenase-2, such as ophthalmic disorders, edemas or neoplasia disorders having an inflammatory component mediated by cyclooxygenase-2.

Carbonic Anhydrase Inhibitors

One aspect of the invention provides a method to treat a carbonic anhydrase mediated disorder comprising administering to a subject a therapeutically effective amount of a carbonic anhydrase inhibitor or pharmaceutically acceptable salt or prodrug thereof corresponding to formula (I)

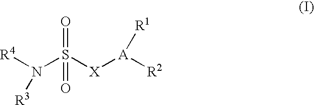

wherein:

X comprises a 5- or 6-membered heterocyclic or carbocyclic ring, the ring atoms being X₁, X₂, X₃, X₄, and X₅ for 5-membered rings and X₁, X₂, X₃, X₄, X₅ and X₆ for 6-membered rings, wherein X₂ is alpha to each of X₁ and X₃, X₃ is alpha to each of X₂ and X₄, X₄ is alpha to each of X₃ and X₅, X₅ is alpha to X₄ and alpha to X₁ if X is a 5-membered ring or to X₆ if X is a 6-membered ring, and X₆, when present, is alpha to each of X₁ and X₅, wherein X₁, X₂, X₃, X₄, X₅ and X₆ are carbon, nitrogen, oxygen or sulfur;

A is selected from the group consisting of a heterocyclyl or a carbocyclic ring;

R¹ is selected from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R¹ is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo or wherein R¹ together with ring A forms one or more heterocyclyl or carbocyclic rings where the heterocyclyl or carbocyclic ring is optionally substituted with a substituent selected from the group consisting of oxo, acyl, hydrocarbyl, substituted hydrocarbyl, cyano, carboxyl, hydroxyl, amino, nitro, aminocarbonyl, aminosulfonyl and halo;

R² is selected from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R² is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo;

R³ is selected from the group consisting of hydrogen, hydrocarbyl and substituted hydrocarbyl; and

R⁴ is selected from the group consisting of hydrogen, hydrocarbyl and substituted hydrocarbyl.

In yet another aspect, the carbonic anhydrase inhibitor or pharmaceutically acceptable salt or prodrug thereof corresponds to formula (II)

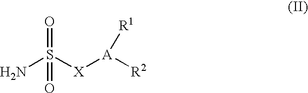

wherein X, A, R¹ and R² are as defined for compounds having formula (I).

In one embodiment for compounds having formula (I) or (II), X₁, X₂, X₃, X₄, and X₅ are selected to provide a 5-membered heterocyclic or carbocyclic ring where each of X¹, X₂, X₃, X₄, and X₅ are carbon, nitrogen, oxygen or sulfur. In one alternative of this embodiment, X¹, X₂, X₃, X₄, and X₅ are selected to provide an optionally substituted cyclopentane or cyclopentene ring. In another alternative of this embodiment, X₁, X₂, X₃, X₄, and X₅ are selected to provide an optionally substituted furan, thiophene, pyrrole, 2H-pyrrole, 3H-pyrrole, pyrazole, 2H-imidazole, 1,2,3-triazole, 1,2,4-triazole, 1,2-dithiole, 1,3-dithiole, 3H-1,2-oxathiole, oxazole, thiazole, isothiazole, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 3H-1,2,3-oxadiazole, 1,2,4-dioxazole, 1,3,2-dioxazole, 1,3,4-dioxazole, 5H-1,2,5-oxathiazole or a 1,3-oxathiole ring.

Alternatively for compounds having formula (I) or (II), X¹, X₂, X₃, X₄, X₅ and X₆ are selected to provide a 6-membered heterocyclic, heteroaromatic, aromatic or carbocyclic ring where each of X¹, X₂, X₃, X₄, X₅, and X₆ are carbon, nitrogen, oxygen or sulfur. In one alternative of this embodiment, X¹, X₂, X₃, X₄, X₅, and X₆ are selected to provide an optionally substituted cyclohexane or cyclohexene ring. In another alternative of this embodiment, X¹, X₂, X₃, X₄, X₅, and X₆ are selected to provide an optionally substituted benzene, 2H-pyran, 4H-pyran, 2-pyrone, 4-pyrone, 1,2-dioxin, 1,3-dioxin, pyridine, pyridazine, pyrimidine, pyrazine, piperazine, 1,3,5-triazine, 1,2,4-triazine, 1,2,3-triazine, 4H-1,2-oxazine, 2H-1,3-oxazine, 6H-1,3-oxazine, 6H-1,2-oxazine, 1,4-oxazine, 2H-1,2-oxazine, 4H-1,4-oxazine, 1,2,5-oxathiazine, 1,4-oxazine, o-isoxazine, p-isoxazine, 1,2,5-oxathiazine, 1,2,6-oxathiazine, 1,4,2-oxadiazine, 1,3,5,2-oxadiazine or a tetrahydro-p-isoxazine ring.

In another embodiment is provided compounds having formula (II) wherein:

X is as defined for any embodiment for compounds having formula (I) or (II);

A is selected from thienyl, oxazolyl, furyl, pyrrolyl, thiazolyl, imidazolyl, isothiazolyl, isoxazolyl, pyrazolyl, cyclopentenyl, phenyl, and pyridyl;

R¹ is selected from the group consisting of heterocyclyl, cycloalkyl, cycloalkenyl and aryl, wherein R¹ is optionally substituted with one or more substituents selected from the group consisting of alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio; and

R² is selected from the group consisting of hydrogen, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl.

In still another aspect, the carbonic anhydrase inhibitor or pharmaceutically acceptable salt or prodrug thereof corresponds to formula (III)

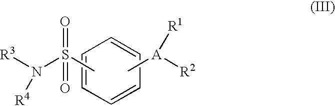

wherein A, R¹, R², R³ and R⁴ are as defined for compounds having formula (I).

In a further aspect, the carbonic anhydrase inhibitor or pharmaceutically acceptable salt or prodrug thereof corresponds to formula (IV)

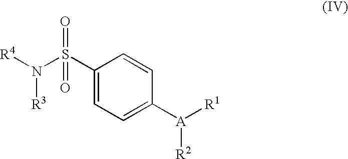

wherein A, R¹, R², R³ and R⁴ are as defined for compounds having formula (I).

One embodiment provides carbonic anhydrase inhibitors corresponding to each of formulas (I) (III), or (IV) wherein:

A is selected from thienyl, oxazolyl, furyl, pyrrolyl, thiazolyl, imidazolyl, isothiazolyl, isoxazolyl, pyrazolyl, cyclopentenyl, phenyl, and pyridyl;

R¹ is selected from the group consisting of heterocyclyl, cycloalkyl, cycloalkenyl and aryl, wherein R¹ is optionally substituted with one or more substituents selected from the group consisting of alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R² is selected from the group consisting of hydrogen, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl;

R³ is hydrogen; and

R⁴ is hydrogen.

In still another embodiment for carbonic anhydrase inhibitors corresponding to any of formulas (I)-(IV), A is selected from thienyl, oxazolyl, furyl, pyrrolyl, thiazolyl, imidazolyl, isothiazolyl, isoxazolyl, pyrazolyl, cyclopentenyl, phenyl, and pyridyl; R¹ and R² are as described for any of the embodiments for compounds having formula (I); and R³, and R⁴ when present, are each hydrogen. In a further alternative of this embodiment, A is pyrazolyl or isoxazolyl. In yet another alternative of this embodiment, A is pyrazolyl. In still another alternative of this embodiment, A is isooxazolyl.

Still another aspect of the invention provides carbonic anhydrase inhibitors or pharmaceutically acceptable salt or prodrug thereof corresponding to formula (V)

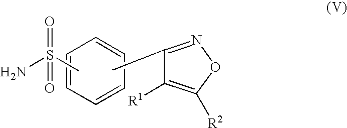

wherein R¹ and R² are as defined for any embodiment for compounds corresponding to formula (I).

Another aspect of the invention provides carbonic anhydrase inhibitors or pharmaceutically acceptable salt or prodrug thereof corresponding to formula (VI)

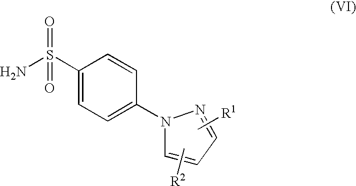

wherein R¹ and R² are as described for any of the embodiments for carbonic anhydrase inhibitors having formula (I).

Yet another aspect of the invention provides carbonic anhydrase inhibitors comprising formula (VII)

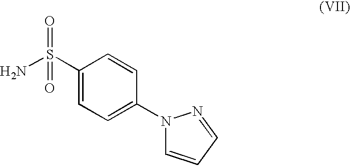

In still another aspect, the carbonic anhydrase inhibitor represented by any of formulas (I)-(VII) is selected from the group of compounds illustrated in Table 1.

TABLE 1

| Compound | |
|---|---|
| No. | Compound |
| 1 | 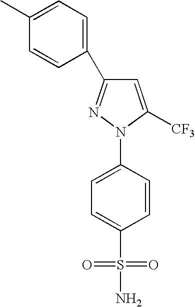 |
| 2 | 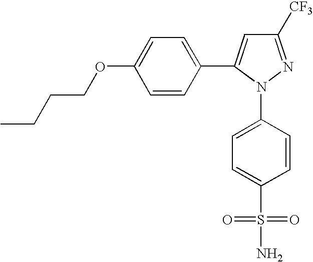 |
| 3 | 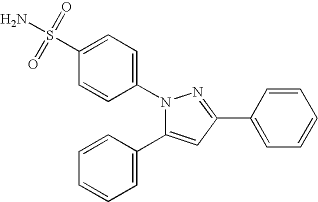 |
| 4 | 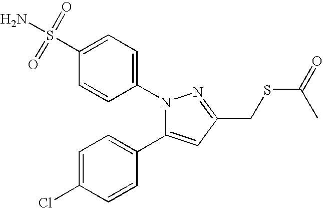 |
| 5 | 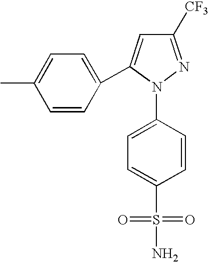 |
| 6 | 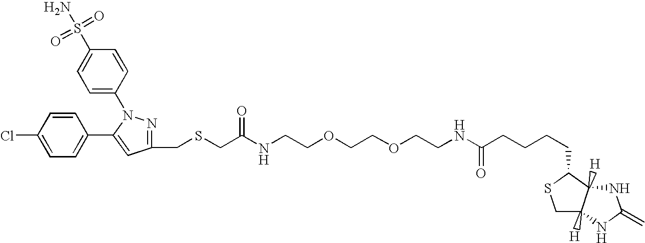 |
| 7 | 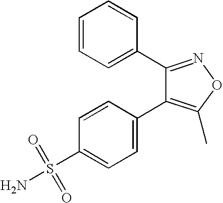 |
| 8 | 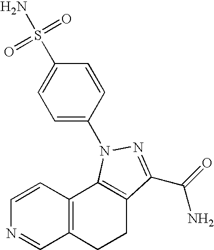 |
| 9 | 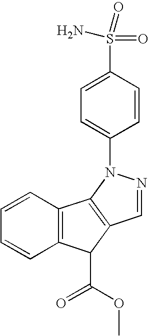 |
| 10 | 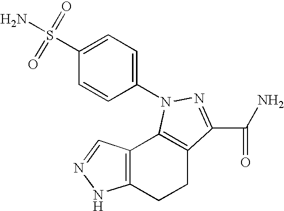 |
| 11 | 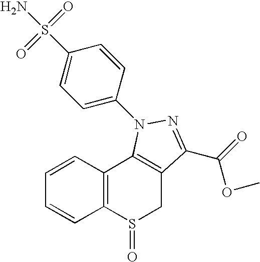 |
| 12 | 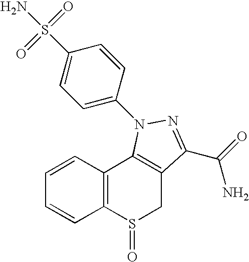 |
| 13 | 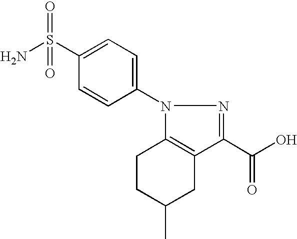 |
| 14 | 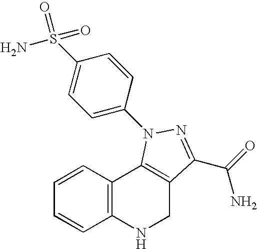 |
| 15 | 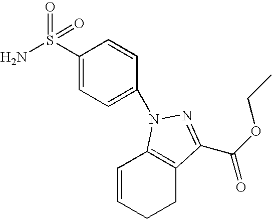 |
| 16 | 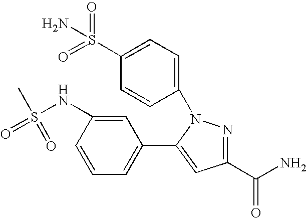 |
| 17 | 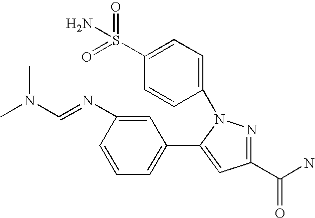 |
| 18 | 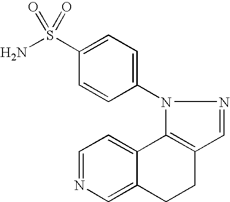 |
| 19 | 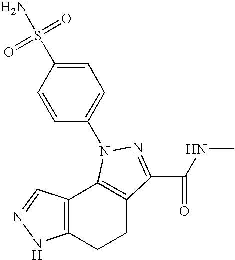 |
| 20 | 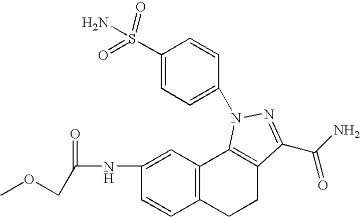 |
| 21 | 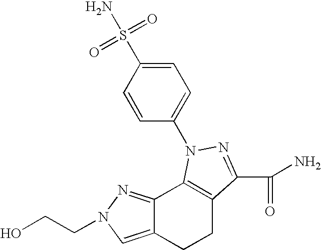 |
| 22 | 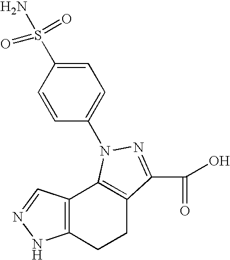 |
| 23 | 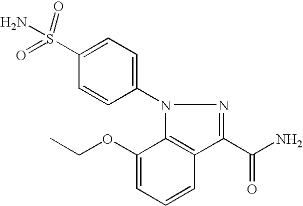 |
| 24 | 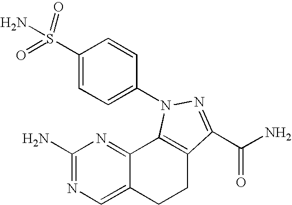 |
| 25 | 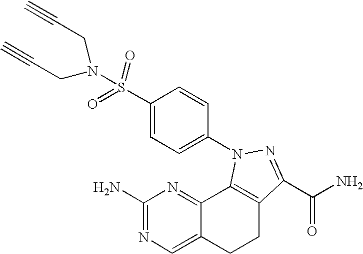 |
| 26 | 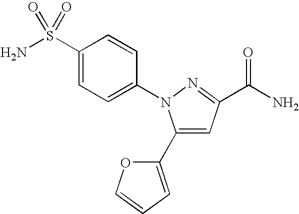 |
| 27 | 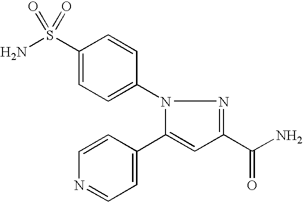 |
| 28 | 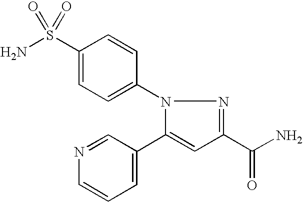 |
| 29 | 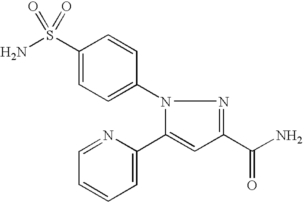 |
| 30 | 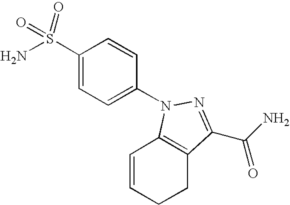 |
| 31 | 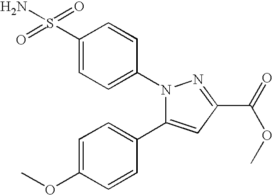 |
| 32 | 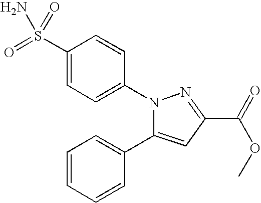 |
| 33 | 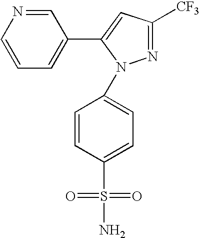 |
| 34 | 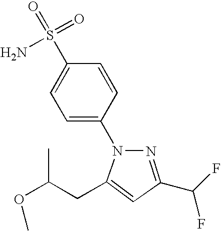 |
| 35 | 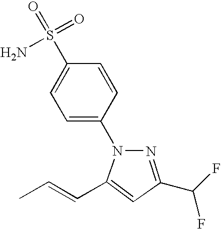 |
| 36 | 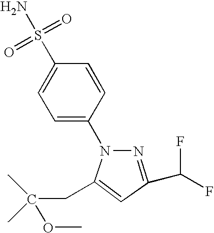 |
| 37 | 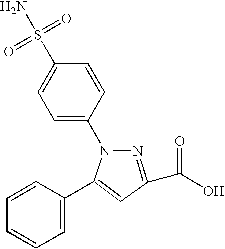 |
| 38 | 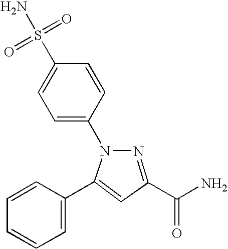 |
| 39 | 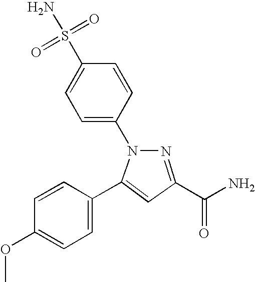 |
| 40 | 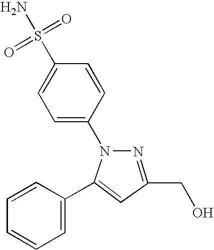 |
| 41 | 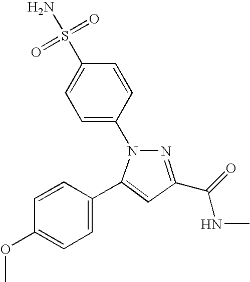 |
| 42 | 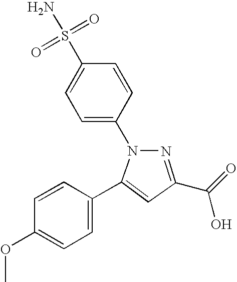 |
| 43 | 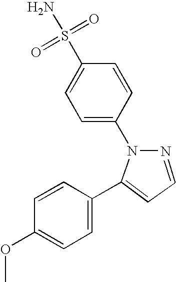 |
| 44 | 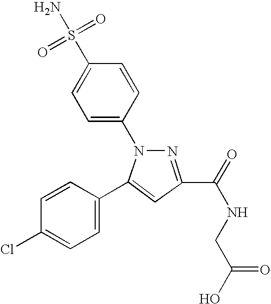 |
| 45 | 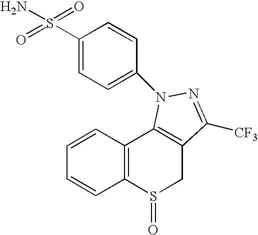 |
| 46 | 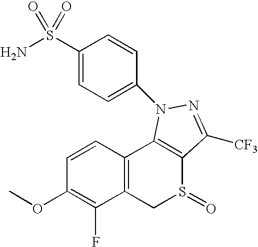 |
| 47 | 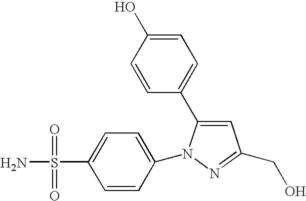 |
| 48 | 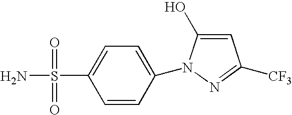 |
| 49 | 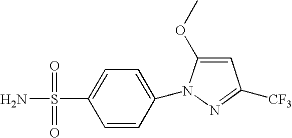 |
| 50 | 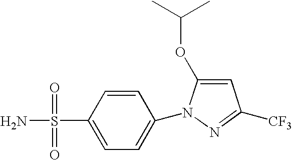 |
| 51 | 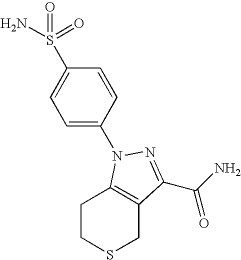 |
| 53 | 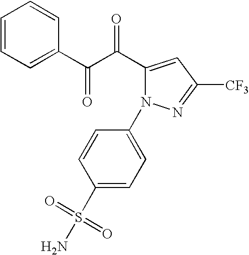 |
| 54 | 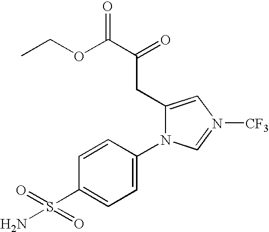 |
| 55 | 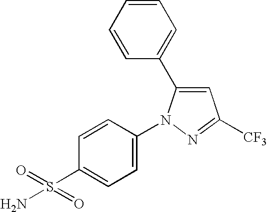 |
| 56 | 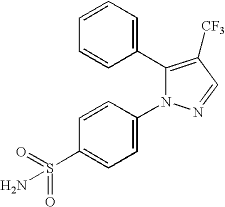 |
| 57 | 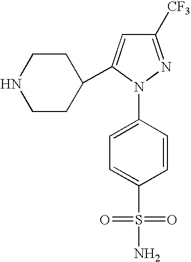 |
| 58 | 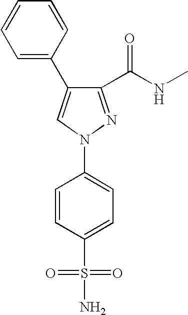 |
| 59 | 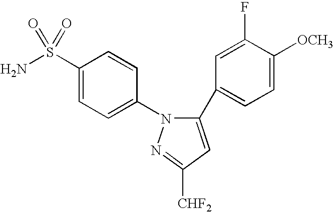 |
| 60 | 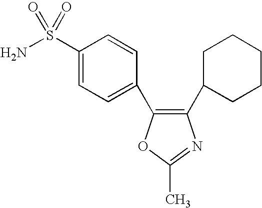 |
| 61 | 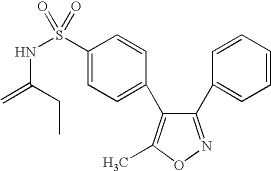 |
| 62 | 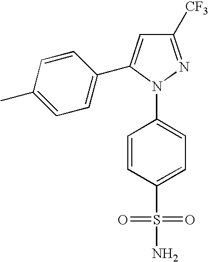 |
| 63 | 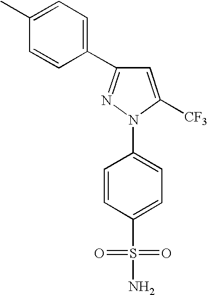 |
| 64 | 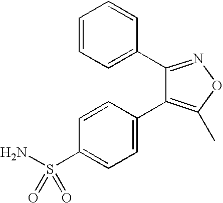 |
| 65 | 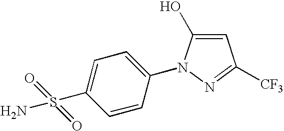 |

The carbonic anhydrase inhibitor employed in the present invention can exist in tautomeric, geometric or stereoisomeric forms. Generally speaking, suitable carbonic anhydrase inhibitors that are in tautomeric, geometric or stereoisomeric forms are those compounds that inhibit carbonic anhydrase activity by about 25%, more typically by about 50%, and even more typically, by about 75% or more when present at a concentration of 100 μM or less. The present invention contemplates all such compounds, including cis- and trans-geometric isomers, E- and Z-geometric isomers, R- and S-enantiomers, diastereomers, d-isomers, l-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures or R and S forms for each stereocenter present.

Indications to be Treated

Generally speaking, the carbonic anhydrase inhibitors having any of formulas (I)-(VII) may be employed to treat or prevent any carbonic anhydrase mediated disorder or related disease in a subject in need of such treatment in which the inhibition of carbonic anhydrase beneficially effects the physiological condition being treated.

Elevated Intraocular Pressure

In some aspects, the invention provides a method for lowering elevated intraocular pressure (IOP) in a subject. A number of ophthalmic disorders are caused in part by deleterious effects resulting from elevated IOP on the optic nerve. The carbonic anhydrase inhibitors corresponding to any of formulas (I)-(VII) may be utilized to treat any ophthalmic disorder in a subject mediated by elevated IOP. By way of example, glaucoma is characterized by a progressive neuropathy caused in part by deleterious effects resulting from increased IOP on the optic nerve. In normal individuals, IOPs range from 12 to 20 mm Hg., averaging approximately 16 mm Hg. At higher values, for instance over 22 mm Hg, there is a risk that the eye may be affected, and if left untreated, result in the formation of glaucoma.

In addition to a carbonic anhydrase inhibitor having any of formulas (I)-(VII), the composition may also comprise a second agent that is an aqueous humor modulating agent. Any aqueous humor modulating agent may be employed to the extent that it lowers IOP. In general, the aqueous humor modulating agent may lower IOP by causing a reduction in the formation of aqueous humor. The aqueous humor modulating agent may also lower IOP by increasing the outflow of aqueous humor from the anterior chamber of the eye. Moreover, the aqueous humor modulating agent may lower IOP by decreasing the inflow of aqueous humor from the anterior chamber of the eye.

In one aspect, the aqueous humor modulating agent is a prostaglandin or a prostaglandin analog. Naturally occurring prostaglandins are C-20 unsaturated fatty acids. Any prostagladin or prostaglandin analog capable of lowering IOP by altering the production, inflow or outflow of aqueous humor may be used in the composition. Suitable prostaglandins that may be employed in the composition include prostaglandin A, prostaglandin B, prostaglandin D, prostaglandin E, prostaglandin F or any combination thereof. Typically, the prostaglandin employed is prostaglandin F or a homolog of prostaglandin F such as PGF₂ₐ. By way of example, PGF₂ₐ is characterized by hydroxyl groups at the C₉ and C₁₁ positions on the alicyclic ring, a cis-double bond between C₅ and C₆, and a trans-double bond between C₁₃ and C₁₄. PGF₂ₐhas the following formula:

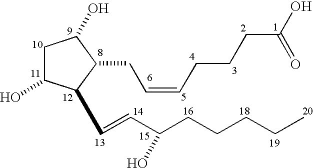

In another embodiment, the aqueous humor modulating agent is a prostaglandin analog. Typically, suitable prostaglandin analogs include any analogs that are similar in structure and function to prostaglandin, which lower IOP. In one alternative of this embodiment, the prostaglandin analog is a prostaglandin FP receptor antagonist. In another alternative of this embodiment, the prostaglandin analog is a prostaglandin F₂ₐ analog. In one embodiment, the prostaglandin F₂ₐ analog is lanaprost. In another embodiment, the F₂ₐ analog is travoprost. In still a further alternative of this embodiment, the prostaglandin analog is unoprostone. In a further alternative of this embodiment, the prostaglandin analog is a prostamide. Generally speaking, the prostamide employed may be any naturally occurring or synthetic prostamide. In one embodiment, the prostamide is the synthetic analog bimatoprost. The preparation and pharmaceutical profiles of several prostaglandin and prostaglandin analogs, including cloprostenol, fluprostenol, latanoprost, and travoprost, are more fully described in U.S. Pat. No. 5,510,383, which is hereby incorporated by reference in its entirety.

In a further aspect, the aqueous humor modulating agent is a beta adrenergic receptor antagonist. Beta adrenergic receptor antagonist bind beta-adrenergic receptors such as the beta₁ adrenergic receptor or the beta₂ adrenergic receptor. By binding to these receptors, the beta adrenergic receptor antagonist decrease the ability of the body's own natural epinephrine to bind to those receptors, leading to inhibition of various processes in the body's sympathetic system, including a reduction in aqueous humor secretion by ciliary tissues in the eye. Generally speaking, any beta adrenergic receptor antagonist capable of lowering IOP by altering the production, inflow or outflow of aqueous humor may be used in the composition. In some embodiments, the beta adrenergic receptor antagonist may be selective for the beta₁ adrenergic receptor. By way of example, suitable selective beta₁ adrenergic receptor antagonist include betaxolol and its enantiomer levobetaxolol. In other embodiments, the beta adrenergic receptor antagonist may be non-selective, blocking both the beta₁ adrenergic receptor and the beta₂ adrenergic receptor. Examples of suitable non-selective beta adrenergic receptor antagonist include timolol, levobunolol, carteolol and metipranolol.

In yet another aspect, the aqueous humor modulating agent is an adrenergic agonist. Adrenergic agonist typically bind to and stimulate adrenergic receptors, causing responses similar to those of adrenaline and noradrenaline, including the inhibition of aqueous humor production. In general, any adrenergic receptor agonist capable of lowering IOP by altering the production, inflow or outflow of aqueous humor may be used in the composition. In one embodiment, the adrenergic receptor agonist is alpha-2 adrenergic receptor agonist. By way of example, suitable alpha-2 adrenergic receptor agonist include apraclonidine and brimonidine. In a further embodiment, the adrenergic receptor agonist is epinephrine. In some embodiments, the adrenergic receptor agonist may be a pharmaceutically acceptable salt of epinephrine such as epinephryl borate, epinephrine bydrochloride or epinephrine bitartate. In other embodiments, the adrenergic receptor agonist may be a prodrug of epinephrine such as dipivefrin.

In still another aspect, the aqueous humor modulating agent is a mitotic. Generally speaking, miotics are divided into two categories: direct and indirect cholinergic agents. Irrespective of their classification, mitotic agents generally lower IOP by stimulating smooth muscle muscarinic receptors, causing a widening of the trabecular meshwork to increase aqueous humor outflow. By way of example, suitable direct cholinergic agents include pilocarpine, pilocarpine hydrochloride, and carbachol. Examples of suitable indirect cholinergic agents include echothiophate iodide, echothiophate, demacarium, and physostigmine.

In a further aspect, the aqueous humor modulating agent is a carbonic anhydrase inhibitor other than a carbonic anhydrase inhibitor having any of formulas (I)-(VII). Carbonic anhydrase is involved in producing bicarbonate, which is required for aqueous humor production by the ciliary tissues in the eye. By inhibiting carbonic anhydrase, accordingly, production of aqueous humor is substantially reduced. Generally speaking, the carbonic anhydrase inhibitor may inhibit any isomer of the metalloprotein enzyme that catalyzes the interconversion of CO₂ and H₂CO₃ (CO₂ +O₂→HCO₂⁻+H⁺). Typically, however, the carbonic anhydrase inhibitor will inhibit the carbonic anhydrase I, carbonic anhydrase II or carbonic anhydrase IV isoform. Examples of suitable carbonic anhydrase inhibitors include acetazolamide, methazolamide, dorzolamide hydrochloride ophthalmic solution, dorzolamide hydrochloride-timolol maleate ophthalmic solution, brinzolamide hydrochloride, dorzolamide, and brinzolamide.

Other aqueous humor modulating agents that may be used to reduce IOP include cannabinoids drug class, for example, anandamine; selective and unselective PKC inhibitors drug class; rho kinase inhibitors drug class; and combinations thereof; corticosteroid receptor antagonist; selective and nonselective dopamine DA-1 agonist; TNF antagonist; somatostatin selective sst4 agonist; angiotensin II antagonist; thyroxine; adenosine 3 antagonist, vacuolar proton ATPase inhibitors such as bafilomycin; sodium hydrogen antiporter inhibitors; chloride anion exchanger inhibitors; and combinations thereof.

It is contemplated that the composition may include more than one aqueous humor modulating agent. Generally speaking, combinations are selected so as to include agents that have different modes of action and work on different receptor sites or enzymes, but that do not antagonize one another. By way of illustrative example, an ineffective combination may include brimonidine with a beta blocker and brimonidine with epinephrine. Both brimonidine and beta blockers suppress the formation of cAMP in the ciliary epithelium, while epinephrine upregulates the adenyl cyclase enzyme that brimonidine indirectly inhibits. By way of further illustrative example, an effective combination may include a beta blocker with a cholinergic agent or a beta blocker with a carbonic anhydrase inhibitor, as both combinations include agents that target different receptor sites or enzymes.

Ophthalmic Disorders

In another embodiment, the carbonic anhydrase mediated disorder is an ophthalmic disorder. Ophthalmic disorders benefited by carbonic anhydrase inhibition include optic neuropathy and cystoid macular edema. In an alternative embodiment, the opthalmic disorder is glaucoma. Any type of glaucoma may be treated with the compounds of the invention. By way of example, the compounds may be utilized to treat open angle glaucoma, acute angle closure glaucoma and secondary glaucoma.

Compounds having any of formulas (I)-(VII) may also be administered with any other drug or agent known in the art to have utility for treating or preventing ophthalmic disorders. In one embodiment, the compounds of the invention may be co-administered with another carbonic anhydrase inhibitor known to be effective in the treatment of ophthalmic disorders, such as acetazolamide, Daranide®, Diamox® or Neptazane®. In an alternative of this embodiment, compounds having formula (I), (II), or (III) are co-administered with osmotic diuretics (e.g. mannitol), miotics (e.g. Isopto Carpine®, Pilocar®, Pilopine®, Pilocarpine® or Ocusert®), epinephrine (e.g. Epifrin® or Propine®), alpha-adrenergic agonist (e.g. Alphagan®, Iopidine®, or Trusopt®), or beta-blockers (e.g. Betagan®, Betiptic®, Ocupress®, Timoptic®, Optipranalol®, or Betimol®) for the treatment of glaucoma. In yet another alternative of this embodiment, compounds having any of formulas (I)-(VII) are co-administered with one or more intraophthalmic or ocular pressure-reducing drugs including, latanoprost (i.e. Xalatan®), travoprost, bimatoprost, or unoprostol for the treatment of glaucoma. In still another alternative of this embodiment, the compounds of the invention are co-administered with another agent such as demulcents, antibiotics, antivirals, steroids, NSAIDs and other anti-inflammatory agents, acetylcholine blocking agents, adrenergic agonist, beta-adrenergic blocking agents and other antiglaucoma agents, antihypertensives, antihistamines, or anticataract agents as a topical application for the treatment of glaucoma. Suitable drugs for use in this embodiment include acebutolol, aceclidine, acetylsalicylic acid (aspirin), acetylsulfisoxazole, alclofenac, alprenolol, amfenac, amiloride, aminocaproic acid, p-aminoclonidine, aminozolamide, anisindione, apafant, atenolol, bacitracin, benoxaprofen, benoxinate, benzofenac, bepafant, betamethasone, betaxolol, bethanechol, brimonidine, bromfenac, bromhexine, bucloxic acid, bupivacaine, butibufen, carbachol, carprofen, cephalexin, chloramphenicol, chlordiazepoxide, chlorprocaine, chlorpropamide, chlortetracycline, cicloprofen, cinmetacin, ciprofloxacin, clidanac, clindamycin, clonidine, clonixin, clopirac, cocaine, cromolyn, cyclopentolate, cyproheptadine, demecarium, dexamethasone, dibucaine, diclofenac, diflusinal, dipivefrin, dorzolamide, enoxacin, eperezolid, epinephrine, erythromycin, eserine, estradiol, ethacrynic acid, etidocaine, etodolac, fenbufen, fenclofenac, fenclorac, fenoprofen, fentiazac, flufenamic acid, flufenisal, flunoxaprofen, fluorocinolone, fluorometholone, flurbiprofen and esters thereof, fluticasone propionate, furaprofen, furobufen, furofenac, furosemide, gancyclovir, gentamycin, gramicidin, hexylcaine, homatropine, hydrocortisone, ibufenac, ibuprofen and esters thereof, idoxuridine, indomethacin, indoprofen, interferons, isobutylmethylxanthine, isofluorophate, isoproterenol, isoxepac, ketoprofen, ketorolac, labetolol, lactorolac, levo-bunolol, lidocaine, linezolid, lonazolac, loteprednol, meclofenamate, medrysone, mefenamic acid, mepivacaine, metaproterenol, methanamine, methylprednisolone, metiazinic, metoprolol, metronidazole, minopafant, miroprofen, modipafant, nabumetome, nadolol, namoxyrate, naphazoline, naproxen and esters thereof, neomycin, nepafenac, nitroglycerin, norepinephrine, norfloxacin, nupafant, olfloxacin, olopatadine, oxaprozin, oxepinac, oxyphenbutazone, oxyprenolol, oxytetracycline, penicillins, perfloxacin, phenacetin, phenazopyridine, pheniramine, phenylbutazone, phenylephrine, phenylpropanolamine, phospholine, pilocarpine, pindolol, pirazolac, piroxicam, pirprofen, polymyxin, polymyxin B, prednisolone, prilocaine, probenecid, procaine, proparacaine, protizinic acid, rimexolone, salbutamol, scopolamine, sotalol, sulfacetamide, sulfanilic acid, sulindac, suprofen, tenoxicam, terbutaline, tetracaine, tetracycline, theophyllamine, timolol, tobramycin, tolmetin, triamcinolone, trimethoprim, trospectomycin, vancomycin, vidarabine, vitamin A, warfarin, zomepirac and pharmaceutically acceptable salts thereof.

Neoplasia Disorders

In still another embodiment, the carbonic anhydrase mediated disorder is a neoplasia disorder or related disease. Compounds having any of formulas (I)-(VII) may be employed to treat or prevent any neoplasia disorder benefited by carbonic anhydrase inhibition. By way of example, these neoplasia disorders or related diseases include renal cancer, leukemia, lung cancer, ovarian cancer, melanoma, colon cancer, cancers of the central nervous system, prostate cancer, cervical cancer and breast cancer.

Compounds having any of formulas (I)-(VII) may also be administered with any other drug or agent known in the art to have utility for treating or preventing neoplasia disorders or related diseases. In one embodiment, the antineoplastic agent is an antimetabolite including folate antagonist (e.g. methotrexate), pyrimidine antagonist (e.g. cytarabine, floxuridine, fludarabine, fluorouracil, or gemcitabine), purine antagonist (e.g. cladribine, mercaptopurine, or thioguanine), and adenosine deaminase inhibitors (e.g. pentostatin). In an alternative embodiment, the antineoplastic agent is an alkylating agent such as chlorambucil, cyclophosphamide, busulfan, ifosfamide, melphalan, or thiotepa. In yet another embodiment, the antineoplastic agent is an akylator agent such as cisplatin, carboplatin, procarbazine, dacarbazine, or altretamine. In still another embodiment, the antineoplastic agent is an anti-tumor antibiotic such as bleomycin, dactinomycin, or mitomycin. In yet a further embodiment, the antineoplastic agent is an immunological agent such as interferon. In another embodiment, the antineoplastic agent is a plant alkaloid including vinca alkaloids (e.g. vinblastine vincristine or vinorelbine), epipodophyllotoxins (e.g. etoposide or teniposide), taxanes (e.g. docetaxel or paclitaxel), and camptothecins (e.g. topotecan or irinotecan). Of course those skilled in the art will appreciate that the particular antineoplastic agents to be administered with a compound having any of formulas (I)-(VII) will vary considerably depending on the type of neoplasia disorder being treated and its stage of progression.

Edema

In yet another embodiment, the carbonic anhydrase mediated disorder or related disease is edema. By way of example, the edema may result from congestive heart failure, liver failure, lung failure or kidney failure, it may be drug induced edema, or it may be a general edema of any part of the subject's body including the heart, brain, eye, lung, vessels, liver, intestines, throat or stomach.

In one embodiment, compounds having any of formulas (I)-(VII) may also be administered with any agent effective for the treatment or prevention of edema. Generally speaking, agents employed in this embodiment are typically diuretics. By way of example, when the edema is associated with lung, liver or heart failure, a suitable diuretic is furosemide (e.g. lasix®). By way of further example, when the edema is associated with glaucoma, a suitable diuretic is acetazolamide. Other suitable diuretics include bumetanide, ethacrynate, torsemide, chlorothiazide, hydrochlorothiazide, indapamide, metolazone, spironolactone, triamterene, amiloride, ethacrynic acid, methazolamide, brinzolamide hydrochloride, dorzolamide, and brinzolamide. A skilled artisan can readily select a suitable diuretic to be co-administered with a carbonic anhydrase inhibitor having any of formulas (I)-(VII) for treatment of specific types of edemas.

Periodic Paralysis

In yet another embodiment, the carbonic anhydrase mediated disorder or related disease is periodic paralysis. Generally speaking, any type of periodic paralysis may be treated by administering to a subject a compound having any of formulas (I)-(VII). In one embodiment, the periodic paralysis is hypokalemic periodic paralysis. In another embodiment, the periodic paralysis is hyperkalemic periodic paralysis.

In addition to a compound having any of formulas (I)-(VII), potassium may also be administered in a combination therapy for the treatment of periodic paralysis.

High Altitude Sickness

In another embodiment, the carbonic anhydrase mediated disorder or related disease is high altitude sickness or mountain sickness. Typically, any type of high altitude sickness may be treated by administering to a subject a compound having any of formulas (I)-(VII). In one embodiment, the high altitude sickness is acute mountain sickness. In another embodiment, the high altitude sickness is moderate mountain sickness. In still another embodiment, the high altitude sickness is severe mountain sickness.

In one embodiment, compounds having any of formulas (I)-(VII) may also be administered with any agent effective for the treatment or prevention of high altitude sickness. Generally speaking, agents employed in this embodiment are typically carbonic anhydrase inhibitors other than compounds having any of formulas (I)-(VII). Suitable carbonic anhydrase inhibitors include acetazolamide, methazolamide, brinzolamide hydrochloride, dorzolamide, and brinzolamide.

Cystine Calculi and Uric Acid Calculi

In still a further embodiment, the carbonic anhydrase mediated disorder is cystine calculi or uric acid calculi. In one alternative of this embodiment, compounds having any of formulas (I)-(VII) may also be administered with any agent effective for the treatment or prevention of cystine calculi or uric acid calculi. Generally speaking, agents employed in this embodiment are typically diuretics. Suitable diuretics include furosemide, bumetanide, ethacrynate, torsemide, chlorothiazide, hydrochlorothiazide, indapamide, metolazone, spironolactone, triamterene, amiloride, ethacrynic acid, acetazolamide, methazolamide, brinzolamide hydrochloride, dorzolamide, and brinzolamide

Cyclooxygenase-2 Mediated Disorders

Moreover, because several compounds having any of formulas (I)-(VII) are cyclooxygenase-2 selective inhibitors in addition to being carbonic anhydrase inhibitors, these compounds may also advantageously be employed to treat or prevent cyclooxygenase-2 mediated conditions. Typical conditions benefited by cyclooxygenase-2 selective inhibition include the treatment or prevention of inflammation, and for treatment or prevention of other inflammation-associated disorders, such as, an analgesic in the treatment of pain and headaches, or as an antipyretic for the treatment of fever. For example, compounds having any of formulas (I)-(VII) are useful to treat or prevent arthritis, including but not limited to rheumatoid arthritis, spondyloarthopathies, gouty arthritis, osteoarthritis, systemic lupus erythematosus and juvenile arthritis. The compounds are also useful in the treatment or prevention of asthma, bronchitis, menstrual cramps, tendinitis, bursitis, skin-related conditions such as psoriasis, eczema, burns and dermatitis, and from post-operative inflammation including ophthalmic surgery such as cataract surgery and refractive surgery. Moreover, the compounds may be employed to treat or prevent gastrointestinal conditions such as inflammatory bowel disease, Crohn's disease, gastritis, irritable bowel syndrome and ulcerative colitis. The compounds may also be employed in treating or preventing inflammation in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury, myocardial ischemia, and the like.

In yet a further embodiment, compounds having any of formulas (I)-(VII) are employed to treat or prevent disorders or related diseases benefited by both carbonic anhydrase and cyclooxygenase-2 inhibition. In addition to any of the indications detailed above, such indications include, but are not limited to, treating ophthalmic or ocular inflammation and more particularly in treatment of ophthalmic diseases such as retinitis, conjunctivitis, retinopathies, uveitis and ophthalmic or ocular photophobia, and of acute injury to eye tissue where there is increased intraocular pressure that responds to treatment with carbonic anhydrase inhibitor drugs or agents. In an alternative of this embodiment, compounds having any of formulas (I)-(VII) are employed for the treatment of corneal graft rejection, ophthalmic or ocular neovascularization, retinal neovascularization including that following injury or infection, diabetic retinopathy, macular degeneration, retrolental fibroplasia and neovascular glaucoma. In yet another alternative of this embodiment, compounds having any of formulas (I)-(VII) are employed to treat or prevent corneal graft rejection, ophthalmic or ocular neovascularization, retinal neovascularization including that following injury or infection, diabetic retinopathy, macular degeneration, retrolental fibroplasia and neovascular glaucoma.

Routes of Administration, Formulations and Dosages

Generally speaking, a compound having any of formulas (I)-(VII) may be administered by any means that will deliver a therapeutically effective dose. Moreover, when administered with another agent as a part of a combination therapy, the agents useful in the practice of the present invention can be formulated into pharmaceutical compositions and administered separately, either simultaneously or sequentially. Alternatively, each agent can be formulated into a single composition comprising both agents. Irrespective of whether both agents are formulated into a single composition or formulated with each agent in a separate composition, the composition may be administered by any means that will deliver a therapeutically effective dose of both agents, as detailed herein or as otherwise known in the art. For example, formulation of agents is discussed in Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Ophthalmic Formulations and Administration

In one aspect, when the composition is employed for the treatment of an ophthalmic disorder, it may be administered directly to the eye by any means known in the art such as in a solution, cream, ointment, emulsion, suspension and slow release formulations. Administration of a composition to the eye generally results in direct contact of the agents with the cornea, through which at least a portion of the administered agents pass. In general, the composition has an effective residence time in the eye of about 2 to about 24 hours, more typically about 4 to about 24 hours and most typically about 6 to about 24 hours.

A composition of the invention can illustratively take the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition may include a gel formulation. In other embodiments, the liquid composition is aqueous. Alternatively, the composition can take the form of an ointment.

In one embodiment, the composition is an aqueous solution, suspension or solution/suspension, which can be presented in the form of eye drops. By means of a suitable dispenser, a desired dosage of each agent can be metered by administration of a known number of drops into the eye. For example, for a drop volume of 25 μl, administration of 1-6 drops will deliver 25-150 μl of the composition. Aqueous compositions of the invention typically contain from about 0.01% to about 50%, more typically about 0.1% to about 20%, still more typically about 0.2% to about 10%, and most typically about 0.5% to about 5%, weight/volume of a compound having any of formulas (I)-(VII).

Generally speaking, aqueous compositions of the invention have ophthalmically acceptable pH and osmolality. “Ophthalmically acceptable” with respect to a formulation, composition or ingredient typically means having no persistent detrimental effect on the treated eye or the functioning thereof, or on the general health of the subject being treated. It will be recognized that transient effects such as minor irritation or a “stinging” sensation are common with topical ophthalmic administration of agents and the existence of such transient effects is not inconsistent with the formulation, composition or ingredient in question being “ophthalmically acceptable” as detailed herein. But formulations, compositions and ingredients employed in the present invention are those that generally cause no substantial detrimental effect, even of a transient nature.

In an aqueous suspension or solution/suspension composition, the agent can be present predominantly in the form of nanoparticles, i.e., solid particles smaller than about 1000 nm in their longest dimension. A benefit of this composition is more rapid release of the agent, and therefore more complete release during the residence time of the composition in a treated eye than occurs with larger particle size. Another benefit is reduced potential for eye irritation by comparison with larger particle size. Reduced eye irritation in turn leads to a reduced tendency for loss of the composition from the treated eye by lacrimation, which is stimulated by such irritation.

In a related composition, the agent typically has a D₉₀ particle size of about 10 to about 2000 nm, wherein about 25% to 100% by weight of the particles are nanoparticles. “D₉₀” is a linear measure of diameter having a value such that 90% by volume of particles in the composition, in the longest dimension of the particles, are smaller than that diameter. For practical purposes a determination of D₉₀ based on 90% by weight rather than by volume is generally suitable.

In one composition, substantially all of the agent particles in the composition are smaller than 100 nm, i.e., the percentage by weight of nanoparticles is 100% or close to 100%. Generally speaking, the average particle size of the agent in this embodiment is typically about 100 to about 800 nm, more typically about 150 to about 600 nm, and even more typically, about 200 to about 400 nm. The agent can be in crystalline or amorphous form in the nanoparticles. Processes for preparing nanoparticles that involve milling or grinding typically provide the agent in crystalline form, whereas processes that involve precipitation from solution typically provide the agent in amorphous form.

The ophthalmic composition in some embodiments can be an aqueous suspension of an agent of low water solubility, wherein typically the agent is present predominantly or substantially entirely in nanoparticulate form. Without being bound by theory, it is believed that release of the agent from nanoparticles is significantly faster than from a typical “micronized” composition having a D₉₀ particle size of, for example, about 10,000 nm or greater.

In another embodiment, an aqueous suspension composition of the invention can comprise a first portion of the agent in nanoparticulate form, to promote relatively rapid release, and a second portion of the agent having a D ₉₀ particle size of about 10,000 nm or greater, that can provide a depot or reservoir of the agent in the treated eye for release over a period of time, for example about 2 to about 24 hours, more typically about 2 to about 12 hours, to promote sustained therapeutic effect and permit a reduced frequency of administration.

In still another embodiment, an aqueous suspension can contain one or more polymers as suspending agents. Useful polymers include water-soluble polymers such as cellulosic polymers, e.g., hydroxypropyl methylcellulose, and water-insoluble polymers such as cross-linked carboxyl-containing polymers.

The composition can be an in situ gellable aqueous solution, suspension or solution/suspension having excipients substantially as disclosed in U.S. Pat. No. 5,192,535, comprising about 0.1% to about 6.5%, typically about 0.5% to about 4.5%, by weight, based on the total weight of the composition, of one or more cross-linked carboxyl-containing polymers. Such an aqueous suspension is typically sterile and has an osmolality of about 10 to about 400 mOsM, typically about 100 to about 250 mOsM, a pH of about 3 to about 6.5, typically about 4 to about 6, and an initial viscosity, when administered to the eye, of about 1000 to about 30,000 cPs, as measured at 25° C. using a Brookfield Digital LVT viscometer with #25 spindle and 13R small sample adapter at 12 rpm. More typically the initial viscosity is about 5000 to about 20,000 cPs. The polymer component has an average particle size not greater than about 50 μm, typically not greater than about 30 μm, more typically not greater than about 20 μm, and most typically about 1 μm to about 5 μm, in equivalent spherical diameter, and is lightly cross-linked to a degree such that, upon contact with tear fluid in the eye, which has a typical pH of about 7.2 to about 7.4, the viscosity of the suspension rapidly increases, to form a gel. This formation of a gel enables the composition to remain in the eye for a prolonged period without loss by lacrimal drainage.

Suitable carboxyl-containing polymers for use in this composition are prepared from one or more carboxyl-containing monoethylenically unsaturated monomers such as acrylic, methacrylic, ethacrylic, crotonic, angelic, tiglic, α-butylcrotonic, α-phenylacrylic, α-benzylacrylic, α-cyclohexylacrylic, cinnamic, coumaric and umbellic acids, most typically acrylic acid. The polymers are cross-linked by using less than about 5%, typically about 0.1% to about 5%, more typically about 0.2% to about 1%, by weight of one or more polyfunctional cross-linking agents such as non-polyalkenyl polyether difunctional cross-linking monomers, e.g., divinyl glycol. Other suitable cross-linking agents illustratively include 2,3-dihydroxyhexa-1,5-diene, 2,5-dimethylhexa-1,5-diene, divinylbenzene, N,N-diallylacrylamide and N,N-diallylmethacrylamide. Divinyl glycol is typically employed. Polyacrylic acid cross-linked with divinyl glycol is called polycarbophil. A polymer system containing polycarbophil is commercially available under the trademark DuraSite® of InSite Vision Inc., Alameda, Calif., as a sustained-release topical ophthalmic delivery system.

In another formulation, the composition can be an in situ gellable aqueous solution, suspension or solution/suspension having excipients substantially as disclosed in U.S. Pat. No. 4,861,760, comprising about 0.1% to about 2% by weight of a polysaccharide that gels when it contacts an aqueous medium having the ionic strength of tear fluid. One such polysaccharide is gellan gum. This composition can be prepared by a procedure substantially as disclosed in U.S. Pat. No. 4,861,760.

In yet another formulation, the composition can be an in situ gellable aqueous solution, suspension or solution/suspension having excipients substantially as disclosed in U.S. Pat. No. 5,587,175, comprising about 0.2% to about 3%, typically about 0.5% to about 1%, by weight of a gelling polysaccharide, typically selected from gellan gum, alginate gum and chitosan, and about 1% to about 50% of a water-soluble film-forming polymer, typically selected from alkylcelluloses (e.g., methylcellulose, ethylcellulose), hydroxyalkylcelluloses (e.g., hydroxyethylcellulose, hydroxypropyl methylcellulose), hyaluronic acid and salts thereof, chondroitin sulfate and salts thereof, polymers of acrylamide, acrylic acid and polycyanoacrylates, polymers of methyl methacrylate and 2-hydroxyethyl methacrylate, polydextrose, cyclodextrins, polydextrin, maltodextrin, dextran, polydextrose, gelatin, collagen, natural gums (e.g., xanthan, locust bean, acacia, tragacanth and carrageenan gums and agar), polygalacturonic acid derivatives (e.g., pectin), polyvinyl alcohol, polyvinylpyrrolidone and polyethylene glycol. The composition can optionally contain a gel-promoting counterion such as calcium in latent form, for example encapsulated in gelatin. This composition can be prepared by a procedure substantially as disclosed in U.S. Pat. No. 5,587,175.

In a further formulation, the composition can be an in situ gellable aqueous solution, suspension or solution/suspension having excipients substantially as disclosed in European Patent No. 0/424,043, comprising about 0.1% to about 5% of a carrageenan gum. In this embodiment, a carrageenan having no more than 2 sulfate groups per repeating disaccharide unit is typical, including kappa-carrageenan, having 18-25% ester sulfate by weight, iota-carrageenan, having 25-34% ester sulfate by weight, and mixtures thereof.

In still another particular formulation, the composition comprises an ophthalmically acceptable mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

In another composition, the agent is solubilized at least in part by an ophthalmically acceptable solubilizing agent. The term “solubilizing agent” generally includes agents that result in formation of a micellar solution or a true solution of the agent. Certain ophthalmically acceptable nonionic surfactants, for example polysorbate 80, can be useful as solubilizing agents, as can ophthalmically acceptable glycols, polyglycols, e.g., polyethylene glycol 400, and glycol ethers.

A class of solubilizing agents suitable for use in solution and solution/suspension compositions of the invention is the cyclodextrins. Suitable cyclodextrins can be selected from α-cyclodextrin, α-cyclodextrin, α-cyclodextrin, alkylcyclodextrins (e.g., methyl-α-cyclodextrin, dimethyl-α-cyclodextrin, diethyl-α-cyclodextrin), hydroxyalkylcyclodextrins (e.g., hydroxyethyl-α-cyclodextrin, hydroxypropyl-α-cyclodextrin), carboxyalkylcyclodextrins (e.g., carboxymethyl-α-cyclodextrin), sulfoalkylether cyclodextrins (e.g., sulfobutylether-α-cyclodextrin), and the like. Ophthalmic applications of cyclodextrins have been reviewed by Rajewski & Stella (1996), Journal of Pharmaceutical Sciences, 85, 1154, at pages 1155-1159. If desired, complexation of an agent by a cyclodextrin can be increased by addition of a water-soluble polymer such as carboxymethylcellulose, hydroxypropyl methylcellulose or polyvinylpyrrolidone, as described by Loftsson (1998), Pharmazie, 53, 733-740.

In some embodiments, one or more ophthalmically acceptable pH adjusting agents or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an ophthalmically acceptable range.

In another embodiment, one or more ophthalmically acceptable salts can be included in the composition in an amount required to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate. Optionally one or more ophthalmically acceptable acids having at least two dissociable hydrogen groups can be included in a polymer-containing composition as interactive agents to retard release of the agent through inhibition of erosion of the polymer, as disclosed in International Patent Publication No. WO 95/03784. Acids useful as interactive agents include boric, lactic, orthophosphoric, citric, oxalic, succinic, tartaric and formic glycerophosphoric acids.

In still another embodiment, an ophthalmically acceptable xanthine derivative such as caffeine, theobromine or theophylline can be included in the composition, substantially as disclosed in U.S. Pat. No. 4,559,343, to reduce ocular discomfort associated with administration of the composition.

In yet another embodiment, one or more ophthalmically acceptable preservatives can be included in the composition to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In a further embodiment, one or more ophthalmically acceptable surfactants, typically nonionic surfactants, can be included in the composition to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

In another embodiment, one or more antioxidants can be included in the composition to enhance chemical stability where required. Suitable antioxidants include ascorbic acid and sodium metabisulfite.

In still another embodiment, one or more ophthalmic lubricating agents can optionally be included in the composition to promote lacrimation or as a “dry eye” medication. Such agents include polyvinyl alcohol, methylcellulose, hydroxypropyl methylcellulose, polyvinylpyrrolidone, etc.

Aqueous suspension compositions of the invention can be packaged in single-dose non-reclosable containers. Such containers can maintain the composition in a sterile condition and thereby eliminate need for preservatives such as mercury-containing preservatives, which can sometimes cause irritation and sensitization of the eye.

Alternatively, multiple-dose reclosable containers can be used, in which case it is typical to include a preservative in the composition.

As a further alternative, the composition can take the form of a solid article that can be inserted between the eye and eyelid or in the conjunctival sac, where it releases the agent as described, for example, in U.S. Pat. Nos. 3,863,633 and 3,868,445, both to Ryde & Ekstedt, incorporated herein by reference. Release is to the lacrimal fluid that bathes the surface of the cornea, or directly to the cornea itself, with which the solid article is generally in intimate contact. Solid articles suitable for implantation in the eye in such fashion are generally composed primarily of polymers and can be biodegradable or non-biodegradable. Biodegradable polymers that can be used in preparation of ocular implants carrying an agent in accordance with the present invention include without restriction aliphatic polyesters such as polymers and copolymers of poly(glycolide), poly(lactide), poly(α-caprolactone), poly(hydroxybutyrate) and poly(hydroxyvalerate), polyamino acids, polyorthoesters, polyanhydrides, aliphatic polycarbonates and polyether lactones. Suitable non-biodegradable polymers include silicone elastomers.

Non Ophthalmic Formulations and Administration

In another aspect, the carbonic anhydrase inhibitors utilized in the present invention may be in the form of free bases or pharmaceutically acceptable acid addition salts thereof as a part of a non ophthalmic formulation. Generally speaking, pharmaceutically-acceptable salts are salts commonly used to form alkali metal salts and to form addition salts of free acids or free bases. The nature of the salt may vary, provided that it is pharmaceutically-acceptable. Suitable pharmaceutically-acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound of any Formula set forth herein.

In addition, the carbonic anhydrase inhibitors can be formulated into pharmaceutical compositions and administered by any means that will deliver a therapeutically effective dose. Generally speaking, suitable routes of administration include any means that results in contact of these compounds with their site of action in the subject's body. More specifically, suitable routes of administration include oral, parenterally, by inhalation spray, rectally, intradermally, transdermally, buccal (i.e. sublingual), or topically in dosage unit formulations containing conventional nontoxic pharmaceutically acceptable carriers, adjuvants, and vehicles as desired. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). Typically, however, the compounds are administered orally.

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of the carbonic anhydrase inhibitor will vary depending upon the patient and the particular mode of administration. In general, the pharmaceutical compositions may contain a carbonic anhydrase inhibitor in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight and most preferably from about 1 to 20 mg/kg body weight, may be appropriate. The daily dose can be administered in one to four doses per day.

Further, when the carbonic anhydrase inhibitor comprises celecoxib, it is preferred that the amount used is within a range of from about 1 to about 20 mg/day.kg, even more preferably from about 1.4 to about 8.6 mg/day.kg, and yet more preferably from about 2 to about 3 mg/day.kg.

When the carbonic anhydrase inhibitor comprises valdecoxib, it is preferred that the amount used is within a range of from about 0.1 to about 5 mg/day.kg, and even more preferably from about 0.8 to about 4 mg/day.kg.

In a further embodiment, when the carbonic anhydrase inhibitor comprises parecoxib, it is preferred that the amount used is within a range of from about 0.1 to about 5 mg/day.kg, and even more preferably from about 1 to about 3 mg/day.kg.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

### EXAMPLES

Materials and Methods:

A reaction mixture consisting of 2 Wibur-Anderson units of human carbonic anhydrase II (Signma, C-6165), 4 mM p-nitrophenol acetate (Sigma, N-8130), 20% DMSO, 0.1 M NA₂SO₄ and 50 mM Tris-HCL, pH 7.6 in a total volume of 200 microliters was prepared. Each inhibitor indicated in Table 2 or 3 below was tested. Final concentrations of each inhibitor ranged from 1 picomolar to 1 micromolar. Control samples that included either no inhibitor or no enzyme were also performed.

The admixture of substrate and enzyme initiates the enzyme reaction. The enzyme activity was monitored by absorbance at 405 nm in a kinetic mode in a plate reader at room temperature and expressed as the change in mOD_{405 nm} per minute. The inhibition at each inhibitor concentration was measured in triplicate. The IC₅₀ value was then derived using the GraphPad Prism curve fitting software. Results for each inhibitor tested are detailed in Table 2 and 3. Table 2 depicts the IC₅₀ expressed as a micromolar concentration of the indicated inhibitor added to the reaction mixture. Table 3 depicts the mean IC₅₀ value expressed as a nanomolar concentration of the indicated inhibitor added to the reaction mixture. The compound number listed in Table 2 and 3 corresponds with the same compound number detailed in Table 1.

TABLE 2

| | | CA Assay | |
|---|---|---|---|
| | | IC₅₀ (μM) and | |
| | | concentration of | Sulfonamide |
| Compound | N | inhibitor added | Structure? |
| 1 | 3 | 0.01 | YES |
| | | (0.015, 0.021, | |
| | | 0.004) | |
| Acetazolamide | 4 | 0.03 | YES |
| | | (0.04, 0.017, | |
| | | 0.03, 0.017) | |
| 2 | 2 | 0.04 | YES |
| | | (0.03, 0.04) | |
| 3 | 1 | 0.04 | YES |
| 4 | 1 | 0.09 | YES |
| 5 | 3 | 0.14 | YES |
| | | (0. 16, 0.15, 0.10) | |
| 6 | 1 | 0.18 | YES |
| 7 | 2 | 0.33 | YES |
| | | (0.4, 0.25) | |
| VIII. | 1 | >100 | NO |
| Rofecoxib/Vioxx | | | |
| IX. | 1 | >100 | NO |
| X. | 1 | >100 | NO |

The structures for compounds VIII, IX, and X as detailed in Table 2 are as follows:

TABLE 3

| | |
|---|---|
| 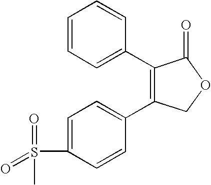 | VIII |
| 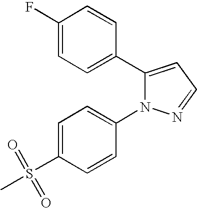 | IX |
| 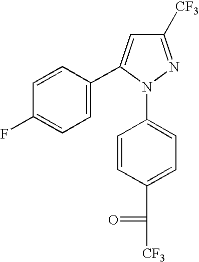 | X |

| | Mean | |
|---|---|---|
| Compound No. | IC₅₀ (nM) | SD |
| Acetazolamide | 9.8 | 2.6 |
| 11 | 3.5 | 1.3 |
| 5 | 48.0 | 20.3 |
| 7 | 79.3 | 12.2 |
| 8 | 195.1 | 11.46 |
| 9 | 33.38 | 6.737 |
| 10 | 45.78 | 13.7 |
| 11 | 209.9 | 25.46 |
| 12 | 465.3 | 49.14 |
| 13 | 70.03 | 13.17 |
| 14 | 101.4 | 0.778 |
| 15 | 5.6 | 1.8 |
| 16 | 33.77 | 8.895 |
| 17 | 181.1 | 23.33 |
| 18 | 251 | 15.91 |
| 19 | 77.65 | 3.783 |
| 20 | 435.1 | 14 |
| 21 | 1224 | 50.2 |
| 22 | 1241 | 106.8 |
| 23 | 202.1 | 43.13 |
| 24 | 60 | 8.704 |
| 25 | 2907 | 116 |
| 26 | 79.36 | 18.02 |
| 27 | 417.9 | 21.64 |
| 28 | 523.2 | 15.13 |
| 29 | 126.7 | 21.92 |
| 30 | 21.95 | 3.899 |
| 31 | 239.5 | 65.27 |
| 32 | 182.1 | 4.525 |
| 33 | 38.65 | 5.035 |
| 34 | 18.4 | 1.296 |
| 35 | 17.99 | 3.918 |
| 36 | 46.26 | 1.534 |
| 37 | 599.3 | 28.78 |
| 38 | 209.8 | 22.84 |
| 39 | 258.4 | 31.04 |
| 40 | 76.75 | 12.62 |
| 41 | 333.6 | 235.4 |
| 42 | 4902 | 399.5 |
| 43 | 202.7 | 41.15 |
| 44 | 80.02 | 10.05 |
| 45 | 43.11 | 10.81 |
| 46 | 60.04 | 10.5 |
| 47 | 67.11 | 12.23 |
| 48 | 81.16 | 13.75 |
| 49 | 8.7 | 3.3 |
| 50 | 17.07 | 3.651 |
| 51 | 35.62 | 18.12 |
| 53 | 10.5 | 5.4 |
| 54 | 10.6 | 3.9 |
| 55 | 25.6 | 5.2 |
| 56 | 109.2 | 30.6 |
| 57 | 138.5 | 48.4 |
| 58 | 388.5 | 58.1 |
| 62 | 48 | 20.3 |
| 63 | 3.5 | 1.3 |
| 64 | 79.3 | 12.2 |
| 65 | 81.15 | 13.8 |

Results:

All Compounds tested containing a sulfonamide inhibited CA II. The selective COX-2 inhibitors, celecoxib and valdecoxib, inhibited CA II activity with IC₅₀s of 0.14 μM and 0.33 μM, respectively. The selective COX-2 inhibitor rofecoxib did not inhibit the enzyme up to 100 μM. The known inhibitor of carbonic anhydrase, acetazolamide, and Compound 1, blocked CA II activity with IC₅₀s of 0.03 μM and 0.01 μM, respectively.

What is claimed is:

1. A method for treating a carbonic anhydrase mediated disorder where the disorder is selected from the group consisting of elevated intraocular pressure, edema, altitude sickness, periodic paralysis, cystine calculi, and uric acid calculi, the method comprising administering to a subject a compound or a pharmaceutically acceptable salt or a prodrug thereof having the structure 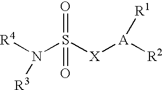
wherein:
X comprises a 5- or 6-membered heterocyclic or carbocyclic ring, the ring atoms being X₁, X₂, X₃, X₄, and X₅ for 5-membered rings and X₁, X₂, X₃, X₄, X₅ and X₆ for 6-membered rings, wherein X₂ is alpha to each of X₁ and X₃, X₃ is alpha to each of X₂ and X₄, X₄ is alpha to each of X₃ and X₅, X₅ is alpha to X₄ and alpha to X₁ if X is a 5-membered ring or to X₆ if X is a 6-membered ring, and X₆, when present, is alpha to each of X₁ and X₅, wherein X₁, X₂, X₃, X₄, X₅ and X₆ are carbon, nitrogen, oxygen or sulfur;
A is selected from the group consisting of a heterocyclyl or a carbocyclic ring;
R¹ is selected from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R¹ is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo or wherein R¹ together with ring A forms one or more heterocyclyl or carbocyclic rings where the heterocyclyl or carbocyclic ring is optionally substituted with a substituent selected from the group consisting of oxo, acyl, hydrocarbyl, substituted hydrocarbyl, cyano, carboxyl, hydroxyl, amino, nitro, aminocarbonyl, aminosulfonyl and halo;
R² is selected from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R² is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo;
R³ is selected from the group consisting of hydrogen, hydrocarbyl and substituted hydrocarbyl; and
R⁴ is selected from the group consisting of hydrogen, hydrocarbyl and substituted hydrocarbyl.

2. The method of claim 1 wherein X is a 5-membered ring selected from the group consisting of cyclopentane, cyclopentenefuran, thiophene, pyrrole, 2H-pyrrole, 3H-pyrrole, pyrazole, 2H-imidazole, 1,2,3-triazole, 1,2,4-triazole, 1,2-dithiole, 1,3-dithiole, 3H-1,2-oxathiole, oxazole, thiazole, isothiazole, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 3H-1,2,3-oxadiazole, 1,2,4-dioxazole, 1,3,2-dioxazole, 1,3,4-dioxazole, 5H-1,2,5-oxathiazole and 1,3-oxathiole.

3. The method of claim 1 wherein X is a 6-membered ring selected from the group consisting of cyclohexane, cyclohexenebenzene, 2H-pyran, 4H-pyran, 2-pyrone, 4-pyrone, 1,2-dioxin, 1,3-dioxin, pyridine, pyridazine, pyrimidine, pyrazine, piperazine, 1,3,5-triazine, 1,2,4-triazine, 1,2,3-triazine, 4H-1,2-oxazine, 2H-1,3-oxazine, 6H-1,3-oxazine, 6H-1,2-oxazine, 1,4-oxazine, 2H-1,2-oxazine, 4H-1,4-oxazine, 1,2,5-oxathiazine, 1,4-oxazine, o-isoxazine, p-isoxazine, 1,2,5-oxathiazine, 1,2,6-oxathiazine, 1,4,2-oxadiazine, 1,3,5,2-oxadiazine, and tetrahydro-p-isoxazine.

4. The method of claim 1 wherein the compound has the structure 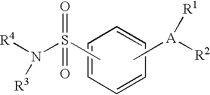
wherein A, R¹, R², R³ and R⁴ are as defined in claim 1.

5. The method of claim 1 wherein the compound has the structure 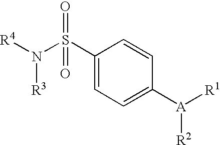
wherein A, R¹, R², R³ and R⁴ are as defined in claim 1.

6. The method of any of claims 1-5
wherein:
A is selected from thienyl, oxazolyl, furyl, pyrrolyl, thiazolyl, imidazolyl, isothiazolyl, isoxazolyl, pyrazolyl, cyclopentenyl, phenyl, and pyridyl;
R¹ is selected from the group consisting of heterocyclyl, cycloalkyl, cycloalkenyl and aryl, wherein R¹ is optionally substituted with one or more substituents selected from the group consisting of alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of hydrogen, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl;
R³ is hydrogen; and
R⁴ is hydrogen.

7. The method of claim 6 wherein A is a pyrazolyl or isoxazolyl ring and R¹, R², R³ and R⁴ are as defined in claim 6.

8. The method of claim 7 wherein A is pyrazolyl.

9. The method of claim 7 wherein A is isoxazolyl.

10. The method of claim 1 wherein the compound has the structure 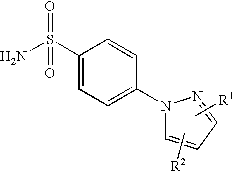
wherein:
R¹ is selected from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R¹ is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo or wherein R¹ together with the pyrazolyl forms one or more heterocyclyl or carbocyclic rings where the heterocyclyl or carbocyclic ring is optionally substituted with a substituent selected from the group consisting of oxo, acyl, hydrocarbyl, substituted hydrocarbyl, cyano, carboxyl, hydroxyl, amino, nitro, aminocarbonyl, aminosulfonyl and halo; and
R² is seleted from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R² is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo.

11. The method of claim 1 wherein the compound has the structure 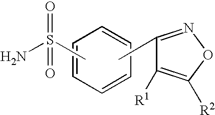
wherein:
R¹ is selected from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R¹ is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo or wherein R¹ together with the pyrazolyl forms one or more heterocyclyl or carbocyclic rings where the heterocyclyl or carbocyclic ring is optionally substituted with a substituent selected from the group consisting of oxo, acyl, hydrocarbyl, substituted hydrocarbyl, cyano, carboxyl, hydroxyl, amino, nitro, aminocarbonyl, aminosulfonyl and halo; and
R² is selected from the group consisting of hydrogen, halo, acyl, carboxyl, hydroxyl, amino, cyano, hydrocarbyl, substituted hydrocarbyl, heterocyclyl, cycloalkyl, cycloalkenyl and aryl, wherein R² is optionally substituted with one or more substituents selected from oxo, acyl, hydrocarbyl, substituted hydrocarbyl, cyano, carboxyl, hydroxyl, amino, nitro, aminocarbonyl, aminosulfonyl and halo.

12. The method of claim 10 or 11 wherein:
R¹ is selected from the group consisting of heterocyclyl, cycloalkyl, cycloalkenyl and aryl, wherein R¹ is optionally substituted with one or more substituents selected from the group consisting of alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio; and
R² is selected from the group consisting of hydrogen, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl.

13. The method of claim 1 wherein the compound is selected from the group consisting of:

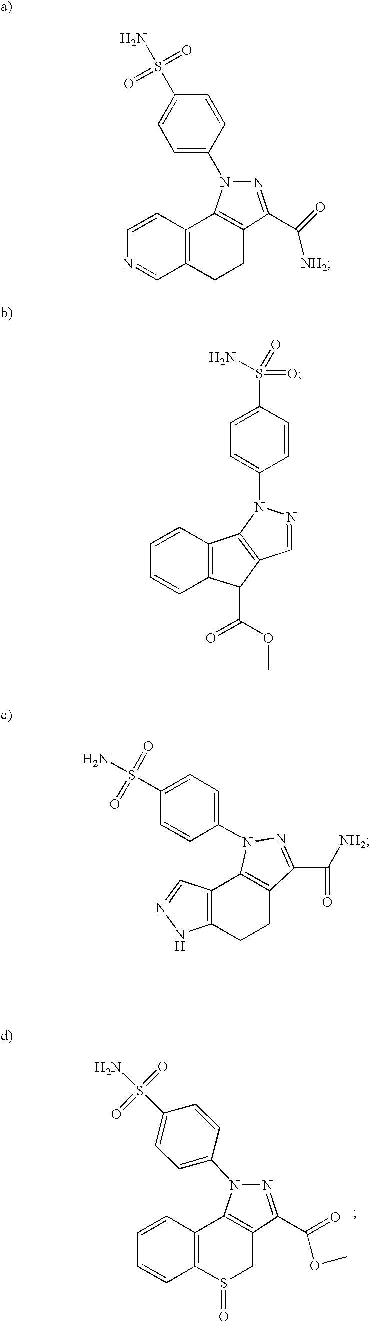

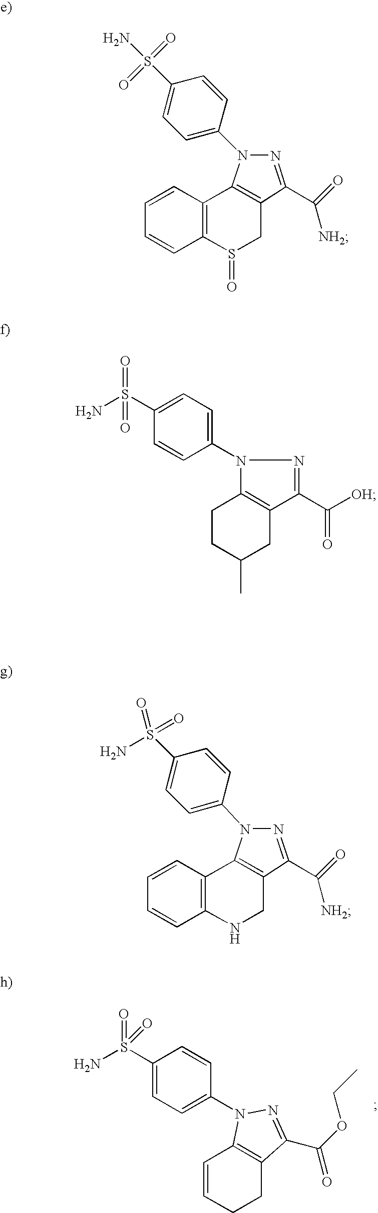

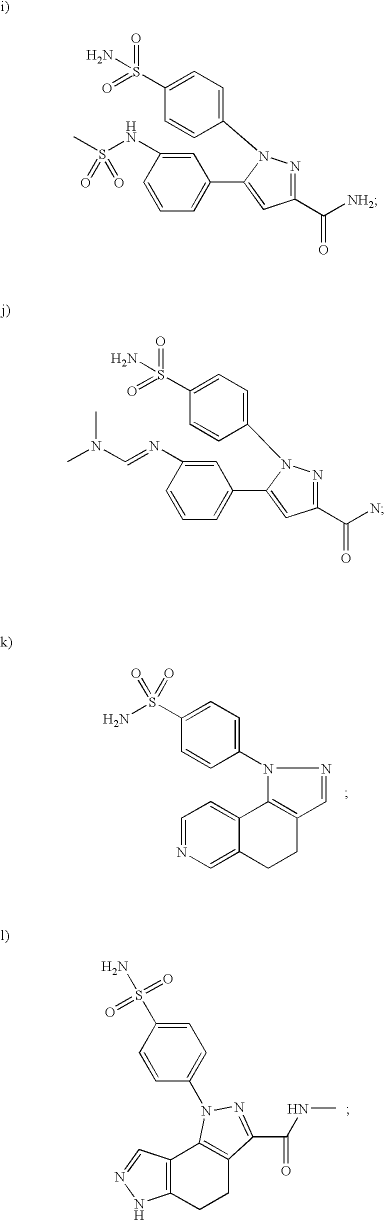

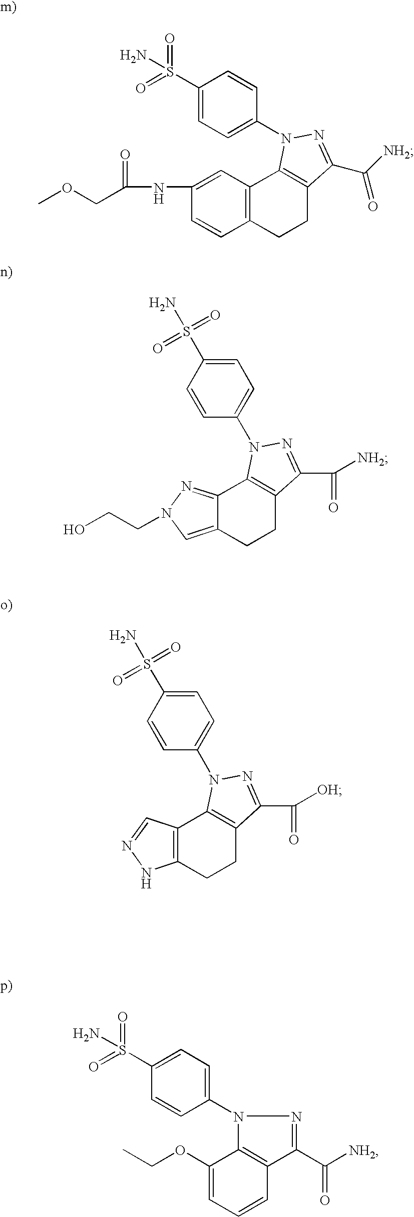

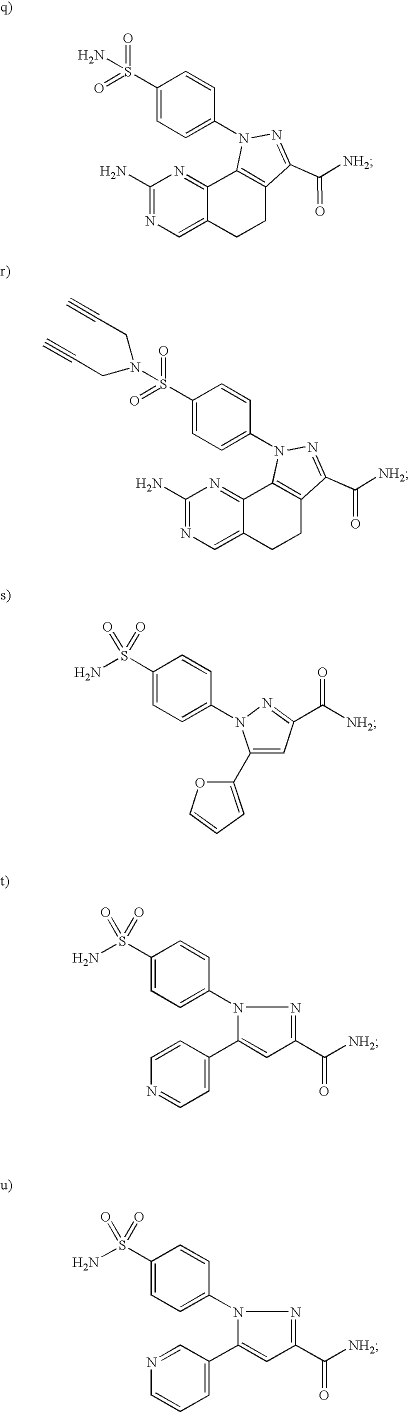

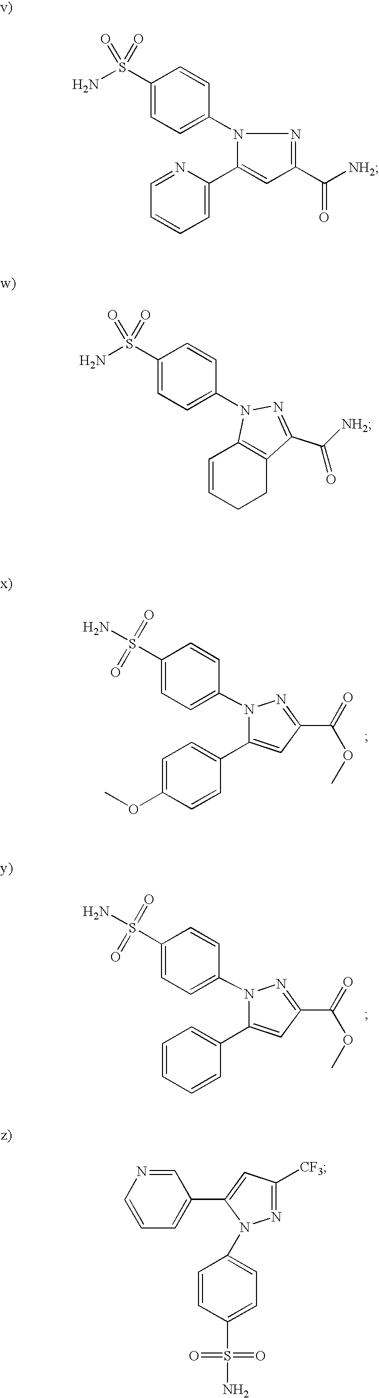

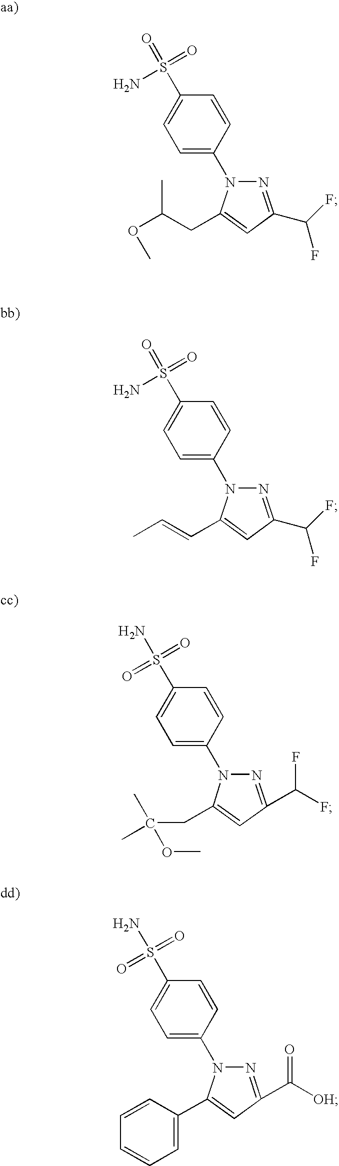

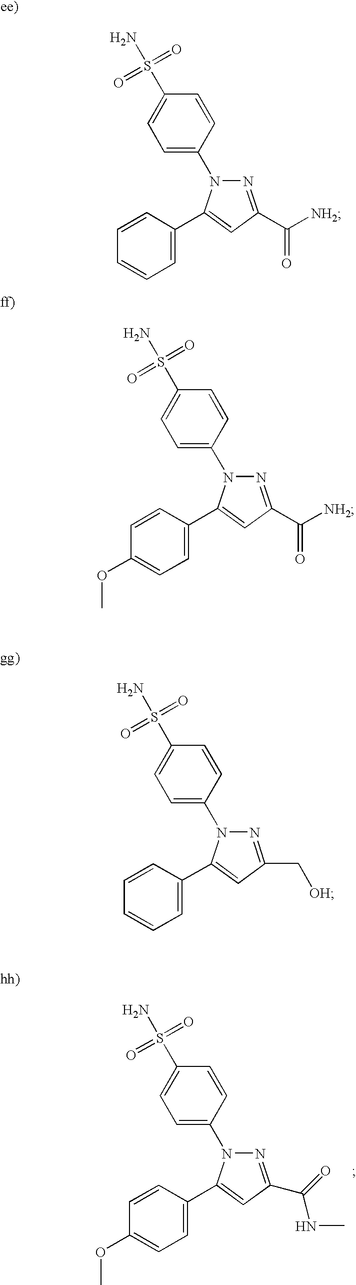

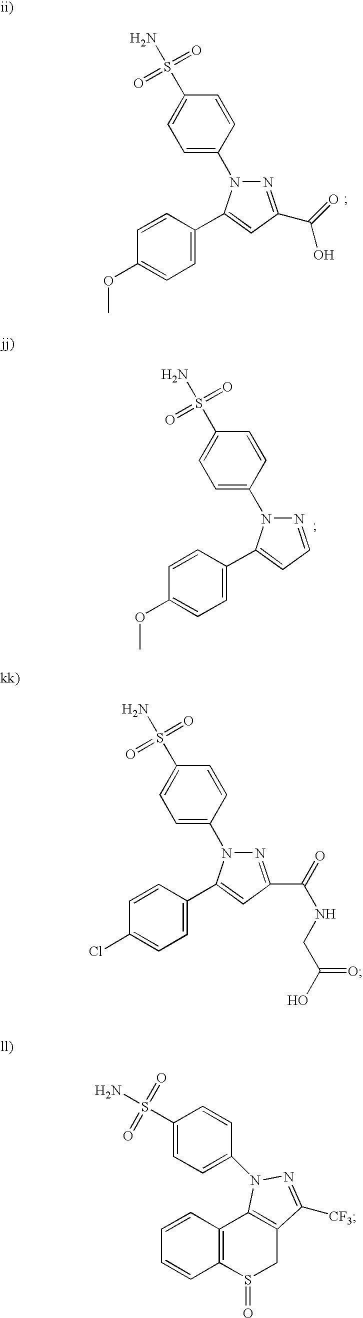

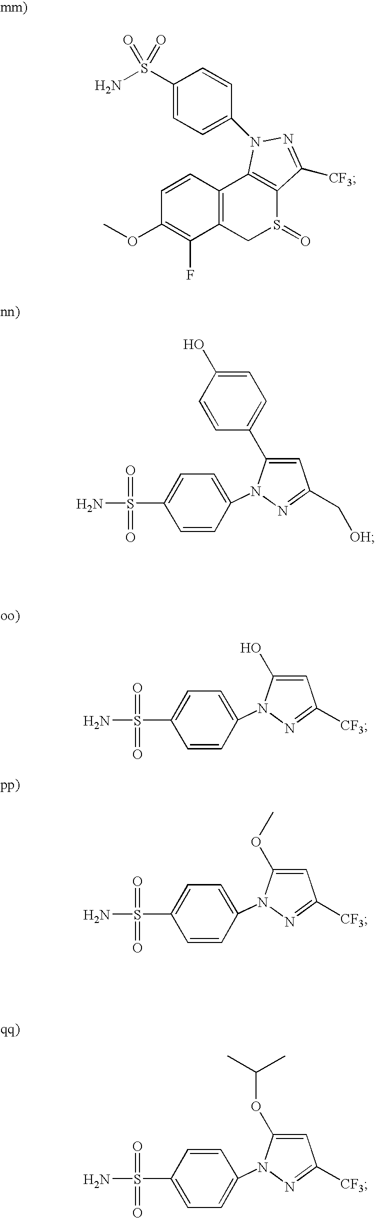

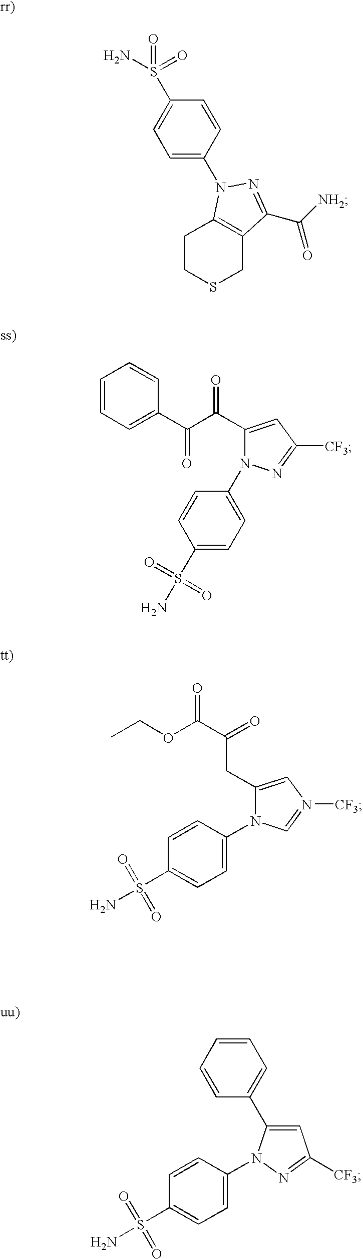

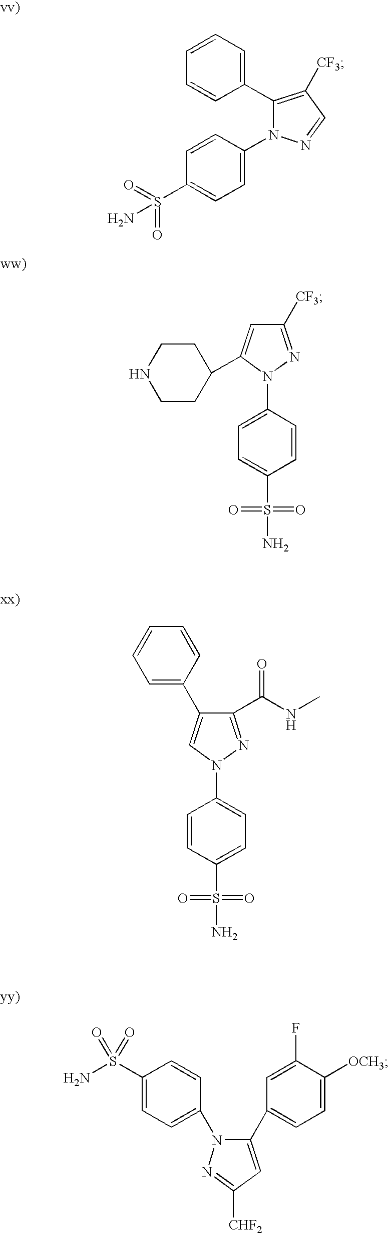

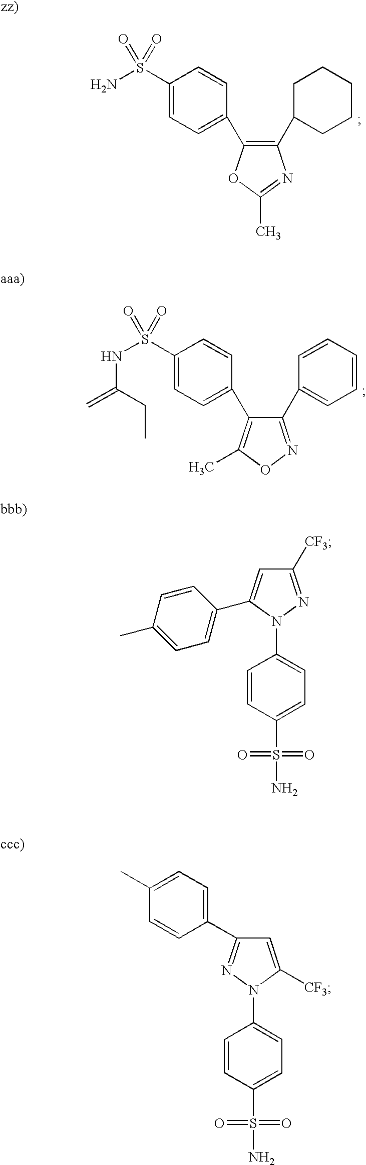

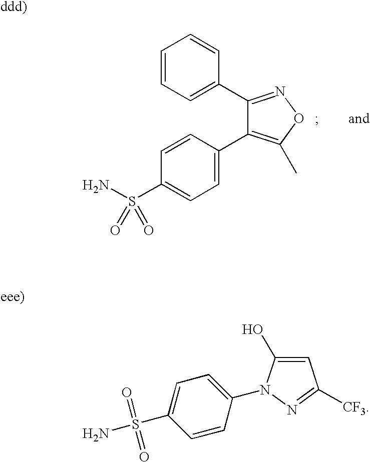

14. A method for treating or preventing a carbonic anhydrase mediated disorder where the disorder is selected from the group consisting of elevated intraocular pressure, edema, altitude sickness, periodic paralyis, cystine calculi, and uric acid calculi, the method comprising administering to a subject a compound or a pharmaceutically acceptable salt or a prodrug thereof having the structure

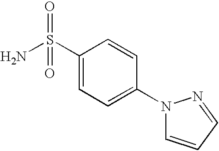

15. The method of claim 1 wherein the carbonic anhydrase mediated disorder is elevated intraocular pressure.

16. The method of claim 15 further comprising administering to the subject a second agent that is an aqueous humor modulating agent.

17. The method of claim 16 wherein the aqueous humor modulating agent reduces the formation of aqueous humor.

18. The method of claim 16 wherein the aqueous humor modulating agent increases outflow of aqueous humor from the anterior chamber of the eye.

19. The method of claim 16 wherein the aqueous humor modulating agent decreases inflow of aqueous humor into the anterior chamber of the eye.

20. The method of claim 16 wherein the aqueous humor modulating agent is a prostaglandin or a prostaglandin analog.

21. The method of claim 20 wherein the aqueous humor modulating agent is a prostaglandin.

22. The method of claim 21 wherein the prostaglandin is selected from prostaglandin A, prostaglandin B, prostaglandin D, prostaglandin E, and prostaglandin F.

23. The method of claim 20 wherein the aqueous humor modulating agent is a prostaglandin analog.

24. The method of claim 23 wherein the prostaglandin analog is a prostaglandin FP receptor antagonist.

25. The method of claim 23 wherein the prostaglandin analog is selected from the group consisting of latanaprost, bimatoprost, unoprostone, and travoprost.

26. The method of claim 16 wherein the aqueous humor modulating agent is a beta adrenergic antagonist.

27. The method of claim 26 wherein the beta adrenergic antagonist is selected from the group consisting of betaxolol, carteolol, levobunolol, metipranolol, timolol, and levobetaxolol.

28. The method of claim 16 wherein the aqueous humor modulating agent is an adrenergic agonist.

29. The method of claim 28 wherein the adrenergic agonist is epinephrine or dipivefrin.

30. The method of claim 16 wherein the aqueous humor modulating agent is a cholinergic agonist.

31. The method of claim 30 wherein the cholinergic agonist is selected from the group consisting of pilocarpine, pilocarpine hydrochloride, carbachol, demacarium, echothiophate idodine, and physostigmine.

32. The method of claim 16 wherein the aqueous humor modulating agent is a carbonic anhydrase inhibitor.

33. The method of claim 32 wherein the carbonic anhydrase inhibitor is a carbonic anhydrase I, II, or IV isosyme inhibitor.

34. The method of claim 32 wherein the carbonic anhydrase inhibitor is selected from the group consisting of acetazolamide, methazolamide, dorzolamide hydrochloride ophthalmic solution, dorzolamide hydrochloride-timolol maleate ophthalmic solution, brinzolamide hydrochloride, dorzolamide, and brinzolamide.

35. The method of claim 1 wherein the carbonic anhydrase mediated disorder is edema.

36. The method of claim 9 wherein the edema is associated with a disorder selected from the group consisting of congestive heart failure, drug induced edema, open angle glaucoma, secondary glaucoma, acute angle closure glaucoma, epilepsy, altitude sickness, familial periodic paralysis, metabolic alkalosis, optic neuropathy, pseudomotor cerebri, and cystoid macular edema.

37. The method of claim 35 further comprising administering to the subject a second agent that is a diuretic.

38. The method of claim 37 wherein the diuretic is selected from the group consisting of furosemide, bumetanide, ethacrynate, torsemide, chlorothiazide, hydrochlorothiazide, indapamide, metolazone, spironolactone, triamterene, amiloride, ethacrynic acid, acetazolamide, methazolamide, brinzolamide hydrochloride, dorzolamide, and brinzolamide.

39. The method of claim 1 wherein the carbonic anhydrase mediated disorder is altitude sickness.

40. The method of claim 39 further comprising administering to the subject a second agent that is a carbonic anhydrase inhibitor selected from the group consisting of acetazolamide, methazolamide, brinzolamide hydrochloride, dorzolamide, and brinzolamide.

41. The method of claim 1 wherein the carbonic anhydrase mediated disorder is periodic paralysis.

42. The method of claim 41 wherein the periodic paralysis is hypokalemic periodic paralysis.

43. The method of claim 41 wherein the periodic paralysis is hyperkalemic periodic paralysis.

44. The method of claim 41 further comprising administering potassium to the subject.

45. The method of claim 1 wherein the carbonic anhydrase mediated disorder is cystine calculi.

46. The method of claim 1 wherein the carbonic anhydrase mediated disorder is uric acid calculi.

47. The method of claim 45 or 46 further comprising administering to the subject a second agent that is a diuretic.

48. The method of claim 48 wherein the diuretic is selected from the group consisting of furosemide, bumetanide, ethacrynate, torsemide, chlorothiazide, hydrochlorothiazide, indapamide, metolazone, spironolactone, triamterene, amiloride, ethacrynic acid, acetazolamide, methazolamide, brinzolamide hydrochloride, dorzolamide, and brinzolamide

49. The method of claim 1 wherein the subject is selected from the group consisting of a human, companion animal, zoo animal and farm animal.

50. The method of claim 49 wherein the subject is a human.
